(19) United States
(12) Patent Application Publication
(10) Pub. No.: US 2023/0228784 A1
(43) Pub. Date: Jul. 20, 2023

(54) SYSTEM, METHOD, AND APPARATUS FOR MANUFACTURE OF ENGINEERED CELLS

(71) Applicant: Multiply Labs Inc., San Francisco, CA (US)

(72) Inventors: Umberto SCARFOGLIERO, Redwood City, CA (US); Ali RIZVI, Menlo Park, CA (US); Andrew BECK, Los Angeles, CA (US); Brigitte SCHMITTLEIN, San Francisco, CA (US); Darius CHAN, San Francisco, CA (US); Dorothy SZYMKIEWICZ, Irvine, CA (US); Federico PARIETTI, San Francisco, CA (US); Kameron CHAN, San Francisco, CA (US); Kelsey POOL, San Francisco, CA (US); Kenny HARSONO, San Francisco, CA (US); Nadia KRECIGLOWA, San Francisco, CA (US); Nolan DICKEY, San Francisco, CA (US); Yasmine AINANE, San Francisco, CA (US)

(21) Appl. No.: 18/154,950

(22) Filed: Jan. 16, 2023

Related U.S. Application Data
(60) Provisional application No. 63/299,799, filed on Jan. 14, 2022.
(60) Provisional application No. 63/334,509, filed on Apr. 25, 2022.

(51) Int. Cl. G01N 35/10; G01N 35/00

(52) U.S. Cl. CPC G01N 35/1067; G01N 35/0099; G01N 35/1081; G01N 2035/00306

(57) ABSTRACT

The present disclosure provides self-centering assemblies, cartridges, pump assemblies, grippers, and other systems, methods, and apparatuses for manufacture of a cell therapy. A gripper includes a gripping unit and a gear unit for connecting and disconnecting connectors, tubes, syringes, or other devices that require twisting of a locking element for a successful fluid connection. In some cases, a gripper also includes a draw/injection unit to draw and inject fluid through the connections. A gripper can be operated by a robotic arm or other versatile manufacturing equipment.

### CROSS-REFERENCE TO RELATED APPLICATION

The present application claims priority to U.S. Provisional Patent Application No. 63/299,799 filed Jan. 14, 2022 and U.S. Provisional Patent Application No. 63/334,509 filed Apr. 25, 2022, each of which is hereby incorporated by reference in its entirety for all purposes.

### TECHNICAL FIELD

The present invention is directed to systems, methods, and apparatuses for manufacture of engineered cells. More particularly, the present invention is directed to systems, methods, and apparatuses for manufacture of cell therapies.

### BACKGROUND

Traditionally, cell therapies and/or capsules are produced with labor-intensive processes. These conventional processes require not only a large number of manufacturing operators, but also the employment of highly skilled (and expensive) technicians. These constraints make it particularly difficult to manufacture cell therapies and/or capsules at an industrial scale.

Given the above-background, what is needed in the art are improved systems, methods, and apparatuses for manufacture of capsules and/or cell therapies.

### SUMMARY

The present disclosure addresses the shortcomings disclosed above by providing systems, methods, and apparatuses for manufacture of cell therapies.

An exemplary embodiment of the device described herein is and/or includes a self-centering assembly including a connection unit and a centering unit. The connection unit includes a holding member. The holding member includes a side wall, and the side wall includes a plurality of contoured surface areas arranged circumferentially. The centering unit is configured to couple the connection unit with a base unit. The centering unit includes a plurality of contoured elastic members arranged circumferentially along an axis of the centering unit, and each respective contoured elastic member in the plurality of elastic members matches a corresponding contoured surface area in the plurality of contoured surface areas on the side wall of the connection unit, thereby allowing the connection unit to be displaced with respect to the axis of the centering unit when the connection unit is subject to an external force and forcing the connection unit to return to its original position when the external force is removed.

In some embodiments, the base unit is an integral part of a device, or is fixedly coupled with the device. In an exemplary embodiment, the device is a cartridge.

In some embodiments, the self-centering assembly includes the base unit.

In some embodiments, the base unit includes a shouldering member, and the holding member of the connection unit sits on the shouldering member of the base unit. In addition, one or more slots are formed at one of the shouldering member of the base unit and the holding member of the connection unit. One or more protrusions are formed at the other of the shouldering member of the base unit and the holding member of the connection unit. The one or more slots and the one or more protrusions are coupled with each other to define a maximal allowed displacement of the connection unit with respect to the base unit.

In some embodiments, the holding member of the connection unit includes a flange sitting on the shouldering member of the base unit. In some embodiments, the one or more slots are formed at the shouldering member of the base unit, and the one or more protrusions are protruded from the flange of the holding member of the connection unit.

In some embodiments, the centering unit includes a plurality of overhangs arranged circumferentially, each sitting on the flange of the holding member of the connection unit to prevent axial movement of the connection unit with respect to the centering unit or the base unit. In an embodiment, the number of the overhangs is the same as the number of contoured elastic members. In another embodiment, the number of the overhangs is different than the number of contoured elastic members.

In some embodiments, the side wall of the holding member of the connection unit further includes a plurality of slots, each corresponding to an overhang in the plurality of overhangs to provide space for the overhang when the connection unit is displaced.

In some embodiments, the centering unit is fixedly coupled with the base unit. In an embodiment, the centering unit is fixedly coupled with the base unit by snap-fitting.

In some embodiments, the side wall is cylindrical.

In some embodiments, the connection unit further includes a fluid fitting member fixedly coupled with the holding member. In an embodiment, the fluid fitting member is a luer-lock connector.

In some embodiments, the holding member of the connection unit includes a first holding piece sitting on the base unit, and a second holding piece fixedly coupled with the first holding piece and configured to secure the fluid fitting member in place with respect to the first holding piece. In an embodiment, the first and second holding pieces are coupled with each other by snap-fitting.

In some embodiments, adjacent contoured surface areas in the plurality of contoured surface areas are spaced apart from each other. In some embodiments, contoured surface areas in the plurality of contoured surface areas are substantially uniformly distributed in a circumferential direction. In some embodiments, the plurality of contoured surface areas includes at least 2, at least 3, at least 4, or at least 5 contoured surface areas. In some embodiments, each contoured surface area is a concave area on the side wall of the holding member of the connection unit.

An exemplary embodiment of the device described herein is and/or includes a self-centering assembly including a connection unit and a centering unit. The connection unit includes a holding member having a cylindrical side wall. The centering unit is configured to couple the connection unit to a device. The centering unit includes a plurality of elastic members arranged circumferentially along an axis of the centering unit. Each of the plurality of elastic members forms a contact with the cylindrical side wall of the holding member of the connection unit, thereby allowing the connection unit to be displaced with respect to the axis of the centering unit when the connection unit is subject to an external force and forcing the connection unit to return to its original position when the external force is removed.

In some embodiments, the holding member of the connection unit includes a flange. The centering unit includes a plurality of overhangs arranged circumferentially, each sitting on the flange of the holding member of the connection unit to prevent axial movement of the connection unit with respect to the centering unit.

In some embodiments, one or more slots are formed at one of the connection unit and the centering unit, and one or more protrusions are formed at the other of the connection unit and the centering unit. The one or more slots and the one or more protrusions are coupled with each other to define a maximal allowed displacement of the connection unit with respect to the centering unit.

In some embodiments, the connection unit further includes a fluid fitting member fixedly coupled with the holding member. In an embodiment, the fluid fitting member is a luer-lock connector.

An exemplary embodiment of the device described herein is and/or includes a cartridge including a cartridge body and one or more connectors. The cartridge body includes one or more first locking members and one or more second locking members, The one or more first locking members and one or more second locking members are substantially orthogonal to each other and configured to lock the cartridge with a gripper or an equipment. The one or more connectors are disposed on the cartridge body and connected to one or more containers secured by the cartridge. Each of the one or more connectors is configured for transferring a fluid to or from a container in the one or more containers.

In some embodiments, the container in the one or more containers is made of a flexible material. In an embodiment, the container in the one or more containers is a bag.

In some embodiments, the container in the one or more containers is made of a rigid material. In an embodiment, the container in the one or more containers is a flask.

In some embodiments, the cartridge body includes a first side and a second side opposite to the first side. The corresponding first locking member in the one or more first locking members and a corresponding second locking member in the one or more second locking members are disposed on each of the first and second sides of the cartridge body. In addition, the corresponding first locking member is substantially orthogonal to the corresponding second locking member.

In an embodiment, for each of the first and second sides of the cartridge body, one of the corresponding first and second locking members includes a cantilevered snap or a spring loaded protrusion, and the other of corresponding first and second locking members includes a slot.

In some embodiments, the cartridge further includes a retaining member for retaining the container, a reference tag, a bar code tag, one or more electronics connectors, one or more additional connectors, one or more sensors, one or more sensor pads, or any combination thereof.

In some embodiments, a respective connector in the one or more connectors is a self-centering assembly disclosed herein.

An exemplary embodiment of the device described herein is and/or includes a cartridge including a cartridge body, a first locking member and one or more locating members. The first locking member is formed or coupled with the cartridge body, and configured to interface with a second locking member of a gripping member of a gripper to lock the cartridge body relative to the gripping member of the gripper in a first direction. The one or more locating members are formed or coupled with the cartridge body, and configured to interface with one or more matching members of the gripping member of the gripper to restrict motion of the cartridge body relative to the gripping member of the gripper in a plane substantially orthogonal to the first direction.

In some embodiments, the cartridge further includes one or more alignment members configured to interface with one or more mating members of a pump assembly. The one or more alignment members allow the cartridge to be loaded to the pump assembly in a direction substantially perpendicular to the first direction.

In some embodiments, the cartridge further includes one or more nesting members configured to nest and secure a flexible tube used in peristalsis.

An exemplary embodiment of the device described herein is and/or includes a gripper for connecting a first unit with a second unit or disconnecting the first unit from the second unit. The first unit includes a first holding member having a first rotation axis. The gripper includes a gripping unit and a gear unit. The gripping unit is operable to engage with the first holding member of the first unit. When the gripping unit is engaged with the first holding member of the first unit, the gripping unit restricts the first holding member of the first unit from moving axially with respect to the gripping unit but allows the first holding member of the first unit to rotate around the first rotation axis. The gear unit includes a first gear operable to rotate the first holding member of the first unit around the first rotation axis. When the first holding member of the first unit rotates around the first rotation axis, the first holding member of the first unit, the gripping unit and the first gear of the gearing unit move axially with respect to the second unit toward or away from the second unit, thereby connecting the first unit with the second unit or disconnecting the first unit from the second unit.

In some embodiments, the first holding member of the first unit includes a first engaging member at an exterior side thereof, and the gripping unit includes a second engaging member at an interior side thereof that matches the first engaging member of the first holding member of the first unit. In an embodiment, one of the first and second engaging members is a groove, and the other of the first and second engaging members is a protrusion that fits with the groove.

In some embodiments, the gripping unit includes one or more gripping members, each rotatable around an axis parallel to the rotation axis of the first holding member of the first unit. In an embodiment, the one or more gripping members includes a first gripping member and a second gripping member symmetrical with respect to each other.

In some embodiments, when the gripping unit is engaged with the first holding member of the first unit, the gripping member defines a gripping axis that coincides with the rotation axis of the first holding member of the first unit.

In some embodiments, the axial movement of the first holding member of the first unit, the gripping unit or the first gear of the gearing unit with respect to the second unit is detected to ensure correct engagement or disengagement of the first and second units. In an embodiment, the detected movement is correlated to angular rotation of a motor to validate the correct engagement or disengagement of the first and second units.

In some embodiments, the first holding member of the first unit includes a first tooth pattern and the first gear of the gear unit includes a second tooth pattern matching the first tooth pattern.

In some embodiments, the first holding member of the first unit includes one or more locating members at a side facing the second unit when the first unit is brought toward the second unit and configured to help locating the first holding member of the first unit with respect to the second unit. In some embodiments, the one or more locating members include a plurality of extrusions disposed circumferentially, thereby assisting in centering the first holding member with respect to the second unit.

In some embodiments, the gripper further includes a capping member configured to constrain translation movement of the first holding member in a plane normal to the rotation axis of the first holding member, translation movement of the first holding member along the rotation axis of the first holding member, or both. In an embodiment, the capping member serves as a hard stop to define a boundary to prevent the gripping unit from closing further inwardly.

In some embodiments, the first unit further including a first fitting member fixedly connected to the first holding member, and the second unit including a second fitting member. Connecting the first unit with the second unit locks the first fitting member with the second fitting member of the second unit, thereby forming a path for transferring a fluid. Disconnecting the first unit from the second unit unlocks the first fitting member from the second fitting member.

In some embodiments, the first fitting member of the first unit is a syringe, a tubing, a connector, or a vessel.

In some embodiments, one of the first fitting member of the first unit and the second fitting member of the second unit includes a male luer lock, and the other of the first fitting member of the first unit and the second fitting member of the second unit includes a female luer lock.

In some embodiments, the second unit is a connection unit of a self-centering assembly disclosed herein.

In some embodiments, the gripper further includes a housing member configured to accommodate the gripping unit, the first gear, a second gear of the gear unit, a capping member, a motor, or a combination thereof.

In some embodiments, the gripper further includes a mounting member for interfacing with an automated equipment. The mounting member and the housing member are axially movable relative to each other.

In some embodiments, the gripper further includes one or more shafts, each fixedly connected to the housing member and extending axially. The mounting member is coupled to the one or more shafts and slidable along the one or more shafts.

In some embodiments, the gripper further includes a covering member to enclose one or more moving parts of the gripper.

In some embodiments, the gripper further includes a draw/injection unit configured to draw or inject a fluid into or from a syringe. The draw/injection unit includes a body gripping member and a carriage member. The body gripping member is configured to interface with a body of the syringe. The carriage member is configured to interface with a plunger of the syringe, and movable relative to the body gripping member in a plunger displacement direction.

In some embodiments, the syringe is the first fitting member of the first unit.

In some embodiments, the draw/injection unit includes an actuating member to actuate a movement of the carriage member. In some embodiments, the actuating member is a screw driven by the first gear of the gear unit.

In some embodiments, the draw/injection unit includes a linear guiding member to guide a movement of the carriage member. The carriage member is coupled with the linear guiding member and movable along the linear guiding member.

In some embodiments, the draw/injection unit includes one or more hard stops, each configured to define a lowest or highest point that the carriage member can travel.

An exemplary embodiment of the device described herein is and/or includes gripper for connecting a first unit with a second unit or disconnecting the first unit from the second unit. The first unit includes a first holding member having a first rotation axis. The gripper includes one or more gripping members and a first gear. The one or more gripping members are operable to engage with the first holding member of the first unit and the second holding member of the second unit. When the one or more gripping members are engaged with the first holding member of the first unit and the second holding member of the second unit, the one or more gripping members (i) restrict axial motion of the first holding member of the first unit relative to the one or more gripping members, (ii) allow rotational motion of the first holding member of the first unit relative to the one or more gripping members around the first rotation axis; and (iii) restrict rotational motion of the second holding member of the second unit relative to the one or more gripping members. The first gear is operable to rotate the first holding member of the first unit around the first rotation axis to connect the first unit with the second unit or disconnect the first unit from the second unit.

In some embodiments, the second unit is a connection unit of a self-centering assembly disclosed herein.

In some embodiments, the first unit includes a first fluid fitting member fixedly coupled with the first holding member. In some embodiments, the first fluid fitting member is a male or female luer connector.

In some embodiments, the one or more gripping members collectively define a gripping axis that coincides with the rotation axis of the first holding member of the first unit.

An exemplary embodiment of the device described herein is and/or includes a method for connecting a first unit with a second unit or disconnecting the first unit from the second unit. The first unit includes a first holding member having a first rotation axis. The method includes positioning, using an automated equipment, a gripper relative to the first holding member of the first unit. The gripper includes a gripping unit and a gear unit, and the gear unit includes a first gear. The method also includes engaging the gripping unit of the gripper with the first holding member of the first unit to restrict the first holding member of the first unit from moving axially with respect to the gripping unit of the gripper but allow the first holding member of the first unit to rotate around the first rotation axis. The method further includes using the first gear of the gear unit of the gripper to rotate the first holding member of the first unit around the first rotation axis. When the first holding member of the first unit rotates around the first rotation axis, the first holding member of the first unit, the gripping unit and the first gear of the gearing unit move axially with respect to the second unit toward or away from the second unit, thereby connecting the first unit with the second unit or disconnecting the first unit from the second unit.

In some embodiments, the automated equipment is a robot.

In some embodiments, the first unit includes a syringe or a connector fixedly coupled with the first holding member.

In some embodiments, the second unit is a connection unit of a self-centering assembly disclosed herein.

An exemplary embodiment of the device described herein is and/or includes a computer system for manufacture of a cell, e.g., a cell of use in cell therapy. The computer system includes one or more processors, and a non-transitory computer-readable medium including computer-executable instructions that, when executed by the one or more processors, cause the processors to perform any method of the present disclosure.

A ninth aspect of the present disclosure provides a non-transitory computer readable storage medium storing one or more programs, the one or more programs including instructions, which when executed by an electronic device with one or more processors and a memory cause the electronic device to execute any method of the present disclosure.

### BRIEF DESCRIPTION OF THE DRAWINGS

The accompanying drawings, which are incorporated into and constitute a part of this specification, illustrate one or more exemplary embodiments of the present disclosure and, together with the Detailed Description, serve to explain the principles and implementations of exemplary embodiments of the invention. The accompanying drawings are not necessarily to scale. The specific design features of the present invention as disclosed herein, including, for example, specific dimensions, orientations, locations, and shapes will be determined in part by the particular intended application and use environment. Reference numbers refer to the same or equivalent parts of the present invention throughout the figure(s) of the drawings. In addition, the components illustrated in the figures are combinable in any useful number and combination

FIG. 1A is a perspective view illustrating an exemplary self-centering assembly in accordance with some embodiments of the present disclosure.

FIG. 1B is an exploded view illustrating the exemplary self-centering assembly of FIG. 1A in accordance with some embodiments of the present disclosure.

FIG. 1C is a perspective view illustrating the exemplary self-centering assembly of FIG. 1A in accordance with some embodiments of the present disclosure.

FIG. 1D is a cross-sectional view illustrating the exemplary self-centering assembly of FIG. 1A in accordance with some embodiments of the present disclosure.

FIG. 1E is an exploded view illustrating an exemplary fluid connector in the exemplary self-centering assembly of FIG. 1A in accordance with some embodiments of the present disclosure.

FIGS. 1F and 1G are perspective views illustrating a component of the exemplary fluid connector of FIG. 1E in accordance with some embodiments of the present disclosure.

FIGS. 1H and 11 are perspective views illustrating another component of the exemplary fluid connector of FIG. 1E in accordance with some embodiments of the present disclosure.

FIG. 1J is a perspective view illustrating some components of the exemplary self-centering assembly of FIG. 1A in accordance with some embodiments of the present disclosure.

FIG. 1K is a perspective view illustrating a portion of FIG. 1J in accordance with some embodiments of the present disclosure.

FIG. 1L is a perspective view illustrating a component of the exemplary self-centering assembly of FIG. 1A in accordance with some embodiments of the present disclosure.

FIG. 1M is a perspective view illustrating some components of the exemplary self-centering assembly of FIG. 1A in accordance with some embodiments of the present disclosure.

FIG. 2A is a perspective view illustrating an exemplary self-centering assembly in accordance with some embodiments of the present disclosure.

FIG. 2B is a perspective view illustrating a component of the exemplary self-centering assembly of FIG. 2A in accordance with some embodiments of the present disclosure.

FIG. 2C is a perspective view illustrating another component of the exemplary self-centering assembly of FIG. 2A in accordance with some embodiments of the present disclosure.

FIG. 3A is a perspective view illustrating an exemplary cartridge in accordance with some embodiments of the present disclosure.

FIG. 3B is another perspective view illustrating the exemplary cartridge of FIG. 3A in accordance with some embodiments of the present disclosure.

FIG. 3C is a cutout view illustrating an implementation of the exemplary cartridge of FIG. 3A in accordance with some embodiments of the present disclosure.

FIG. 3D is a perspective view another implementation of the exemplary cartridge of FIG. 3A in accordance with some embodiments of the present disclosure.

FIG. 4A is a perspective view illustrating an exemplary gripper in accordance with some embodiments of the present disclosure.

FIG. 4B is a cross-sectional view illustrating the exemplary gripper of FIG. 4A in accordance with some embodiments of the present disclosure.

FIG. 4C is an enlarged view of FIG. 4B (partial).

FIG. 4D is a perspective view illustrating the exemplary gripper of FIG. 4A (without a covering member) in accordance with some embodiments of the present disclosure.

FIG. 4E is another cross-sectional view illustrating the exemplary gripper of FIG. 4A in accordance with some embodiments of the present disclosure.

FIG. 4F is a perspective view illustrating an exemplary gripping unit of the exemplary gripper of FIG. 4A in accordance with some embodiments of the present disclosure.

FIG. 4G is a perspective view illustrating an exemplary gripping unit of the exemplary gripper of FIG. 4A, in which the gripping unit of the exemplary gripper engages with a holding member, in accordance with some embodiments of the present disclosure.

FIG. 4H is a perspective view illustrating some components of the exemplary gripper of FIG. 4A in accordance with some embodiments of the present disclosure.

FIG. 4I is a cross-sectional view illustrating the exemplary gripper of FIG. 4A (partial) in accordance with some embodiments of the present disclosure.

FIGS. 4J, 4K and 4L are perspective views illustrating an exemplary operation of an exemplary draw/injection unit of the exemplary gripper of FIG. 4A in accordance with some embodiments of the present disclosure.

FIGS. 4M, 4N, 40 and 4P are perspective views illustrating an exemplary draw/injection unit of the exemplary gripper of FIG. 4A in accordance with some embodiments of the present disclosure.

FIG. 4Q is a perspective view illustrating the exemplary gripper of FIG. 4A in accordance with some embodiments of the present disclosure.

FIG. 4R is a cross-section view illustrating the exemplary gripper of FIG. 4A (partial) in accordance with some embodiments of the present disclosure.

FIG. 4S is a perspective view illustrating some components of the exemplary gripper of FIG. 4A in accordance with some embodiments of the present disclosure.

FIG. 4T is a cross-sectional view illustrating the exemplary gripper of FIG. 4A (partial) in accordance with some embodiments of the present disclosure.

FIG. 4U is a disassembled view illustrating an exemplary unit to be connected or disconnected by a gripper in accordance with some embodiments of the present disclosure.

FIG. 4V is an assembled view illustrating the exemplary unit of FIG. 4U.

FIG. 5A is a perspective view illustrating an exemplary gripper in accordance with some embodiments of the present disclosure.

FIG. 5B is a perspective view illustrating the exemplary gripper of FIG. 5A (without a covering member) in accordance with some embodiments of the present disclosure.

FIG. 5C is a cross-sectional view illustrating the exemplary gripper of FIG. 5A in accordance with some embodiments of the present disclosure.

FIG. 5D is another perspective view illustrating the exemplary gripper of FIG. 5A in accordance with some embodiments of the present disclosure.

FIG. 5E is a cross-sectional view of FIG. 5D.

FIG. 5F is an enlarged view of FIG. 5E (partial).

FIG. 5G is a perspective view illustrating an exemplary gripping unit of the exemplary gripper of FIG. 5A in accordance with some embodiments of the present disclosure.

FIG. 5H is a perspective view illustrating an exemplary gripping unit of the exemplary gripper of FIG. 5A, in which the gripping unit of the exemplary gripper engages with a holding member, in accordance with some embodiments of the present disclosure.

FIG. 5I is a cross-sectional view illustrating the exemplary gripper of FIG. 5A (partial) in accordance with some embodiments of the present disclosure.

FIG. 5J is a perspective view illustrating some components of the exemplary gripper of FIG. 5A in accordance with some embodiments of the present disclosure.

FIG. 5K is a disassembled view illustrating an exemplary unit to be connected or disconnected by a gripper in accordance with some embodiments of the present disclosure.

FIG. 6A is a perspective view illustrating an exemplary clip in accordance with some embodiments of the present disclosure.

FIG. 6B is a perspective view illustrating the exemplary clip of FIG. 6A, in which the gripping clip engages with a holding member, in accordance with some embodiments of the present disclosure.

FIG. 6C is a top view illustrating the exemplary clip of FIG. 6A, in which the gripping clip engages with a holding member in a gripper, in accordance with some embodiments of the present disclosure.

FIG. 6D is a perspective view illustrating another exemplary clip in accordance with some embodiments of the present disclosure.

FIG. 6E is a cross-sectional view illustrating the exemplary clip of FIG. 6D in accordance with some embodiments of the present disclosure.

FIGS. 7A and 7B are perspective views illustrating an exemplary pump assembly in accordance with some embodiments of the present disclosure.

FIG. 7C is a side view illustrating the exemplary pump assembly of FIG. 7A in accordance with some embodiments of the present disclosure.

FIGS. 8A and 8B are perspective views illustrating an exemplary cartridge in accordance with some embodiments of the present disclosure.

FIG. 8C is an assembled view illustrating the exemplary cartridge of FIG. 8A engaged with a gripping member in accordance with some embodiments of the present disclosure.

FIG. 8D is a cross-sectional view illustrating the exemplary cartridge of FIG. 8A engaged with a gripping member in accordance with some embodiments of the present disclosure.

FIG. 8E is a disassembled view illustrating the exemplary cartridge of FIG. 8A disengaged from a gripping member in accordance with some embodiments of the present disclosure.

FIG. 8F is an assembled view illustrating the exemplary cartridge of FIG. 8A engaged with a pump assembly in accordance with some embodiments of the present disclosure.

FIG. 8G is a disassembled view illustrating the exemplary cartridge of FIG. 8A disengaged from a pump assembly in accordance with some embodiments of the present disclosure.

FIG. 8H is a top view illustrating the exemplary cartridge of FIG. 8A engaged with a pump assembly in accordance with some embodiments of the present disclosure.

FIG. 8I is an enlarged view of FIG. 8H (partial).

FIG. 9 is a perspective view illustrating an exemplary gripper in accordance with some embodiments of the present disclosure.

FIGS. 10A, 10B and 10C are perspective views illustrating alternative use of a gripper in accordance with some embodiments of the present disclosure.

FIG. 11A is an assembled view illustrating an exemplary self-centering assembly in accordance with some embodiments of the present disclosure.

FIG. 11B is a disassembled view illustrating the exemplary self-centering assembly of FIG. 11A in accordance with some embodiments of the present disclosure.

FIG. 12 is a block diagram illustrating an exemplary computer system in accordance with some embodiments of the present disclosure.

FIG. 13 is a flowchart illustrating an exemplary method for connecting or disconnecting units in accordance with some embodiments of the present disclosure.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

The description herein, for purpose of explanation, is described with reference to specific implementations. However, the illustrative discussions are not intended to be exhaustive or to limit the implementations to the precise forms disclosed. Many modifications and variations are possible in view of the disclosed teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

For convenience in explanation and accurate definition in the appended claims, the terms “upper,” “lower,” “up,” “down,” “upwards,” “downwards,” “inner,” “outer,” “inside,” “outside,” “inwardly,” “outwardly,” “interior,” “exterior,” “front,” “rear,” “back,” “forwards,” and “backwards,” are used to describe features of the exemplary embodiments with reference to the positions of such features as displayed in the figures.

In addition, although the terms first, second, etc. may be used herein to describe various elements, these elements should not be limited by these terms. These terms are only used to distinguish one element from another. For instance, a first unit could be termed a second unit, and, similarly, a second unit could be termed a first unit, without departing from the scope of the present disclosure. The first unit and the second unit are both units, but they are not the same unit.

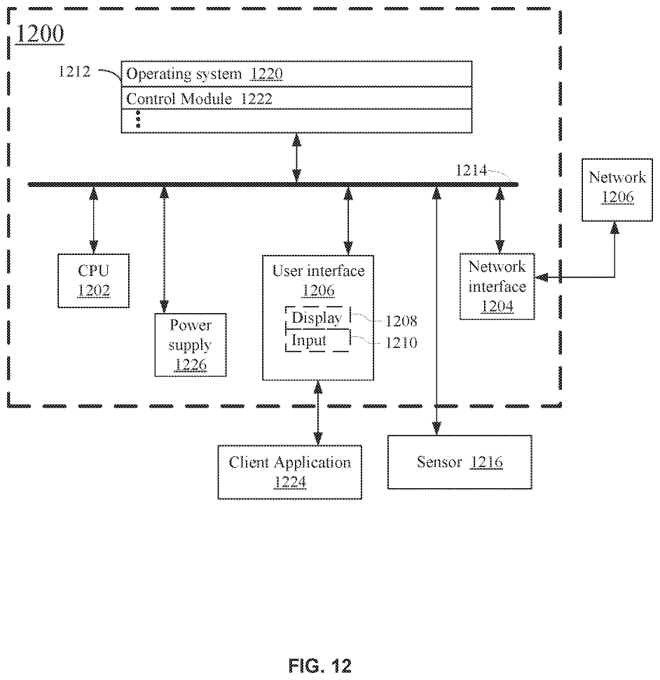

In the present disclosure, unless expressly stated otherwise, descriptions of devices and systems will include implementations of one or more computers. For instance, and for purposes of illustration in FIG. 12, a computer system 1200 is represented as single device that includes all the functionality of the computer system 1200. However, the present disclosure is not limited thereto. For instance, in some embodiments, the functionality of the computer system 1200 is spread across any number of networked computers and/or reside on each of several networked computers and/or by hosted on one or more virtual machines and/or containers at a remote location accessible across a communications network (e.g., communications network 1206 of FIG. 12). One of skill in the art will appreciate that a wide array of different computer topologies is possible for the computer system 1200, and other devices and systems of the preset disclosure, and that all such topologies are within the scope of the present disclosure. Moreover, rather than relying on a physical communications network 1206, the illustrated devices and systems may wirelessly transmit information between each other. As such, the exemplary topology shown in FIG. 12 merely serves to describe the features of an embodiment of the present disclosure in a manner that will be readily understood to one of skill in the art.

FIG. 12 depicts a block diagram of a distributed computer system (e.g., computer system 1200) according to some embodiments of the present disclosure. The computer system 1200 at least facilitates communicating one or more instructions for fabricating a capsule.

In some embodiments, the communication network 1206 optionally includes the Internet, one or more local area networks (LANs), one or more wide area networks (WANs), other types of networks, or a combination of such networks.

Examples of communication networks 1206 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, the computer system 1200 includes one or more processing units (CPUs) 1202, a network or other communications interface 1204, and memory 1212.

In some embodiments, the computer system 1200 includes a user interface 1206. The user interface 1206 typically includes a display 1208 for presenting media. In some embodiments, the display 1208 is integrated within the computer systems (e.g., housed in the same chassis as the CPU 1202 and memory 1212). In some embodiments, the computer system 1200 includes one or more input device(s) 1210, which allow a subject to interact with the computer system 1200. In some embodiments, input devices 1210 include a keyboard, a mouse, and/or other input mechanisms. Alternatively, or in addition, in some embodiments, the display 1208 includes a touch-sensitive surface (e.g., where display 1208 is a touch-sensitive display or computer system 1200 includes a touch pad).

In some embodiments, the computer system 1200 presents media to a user through the display 1208. Examples of media presented by the display 1208 include one or more images, a video, audio (e.g., waveforms of an audio sample), or a combination thereof. In typical embodiments, the one or more images, the video, the audio, or the combination thereof is presented by the display 1208 through a client application (e.g., client application 1224 of FIG. 12). In some embodiments, the audio is presented through an external device (e.g., speakers, headphones, input/output (I/O) subsystem, etc.) that receives audio information from the computer system 1200 and presents audio data based on this audio information. In some embodiments, the user interface 1206 also includes an audio output device, such as speakers or an audio output for connecting with speakers, earphones, or headphones.

Memory 1212 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 1212 may optionally include one or more storage devices remotely located from the CPU(s) 1202. Memory 1212, or alternatively the non-volatile memory device(s) within memory 1212, includes a non-transitory computer readable storage medium. Access to memory 1292 by other components of the computer system 1200, such as the CPU(s) 1212, is, optionally, controlled by a controller. In some embodiments, memory 1212 can include mass storage that is remotely located with respect to the CPU(s) 1202. In other words, some data stored in memory 1212 may in fact be hosted on devices that are external to the computer system 1200, but that can be electronically accessed by the computer system 1200 over an Internet, intranet, or other form of network 1206 or electronic cable using communication interface 1204.

In some embodiments, the memory 1202 of the computer system 1200 stores:

an operating system 1220 (e.g., ANDROID, iOS, DARWIN, RTXC, LINUX, UNIX, OS X, WINDOWS, or an embedded operating system such as VxWorks) that includes procedures for handling various basic system services;

an electronic address associated with the computer system 1200 that identifies the computer system 1200 (e.g., within the communication network 1206);

a control module 1222 for controlling one or more instruments (e.g., an apparatus for a manufacture of a capsule and/or cell therapy) associated with the computer system 1200; and

a client application 1220 for presenting information (e.g., media) using a display 1278 of the computer system 1200.

As indicated above, an electronic address 1204 is associated with the computer system 1200. The electronic address 1214 is utilized to at least uniquely identify the computer system 1200 from other devices and components of the distributed system 1200, such as other devices having access to the communications network 1206.

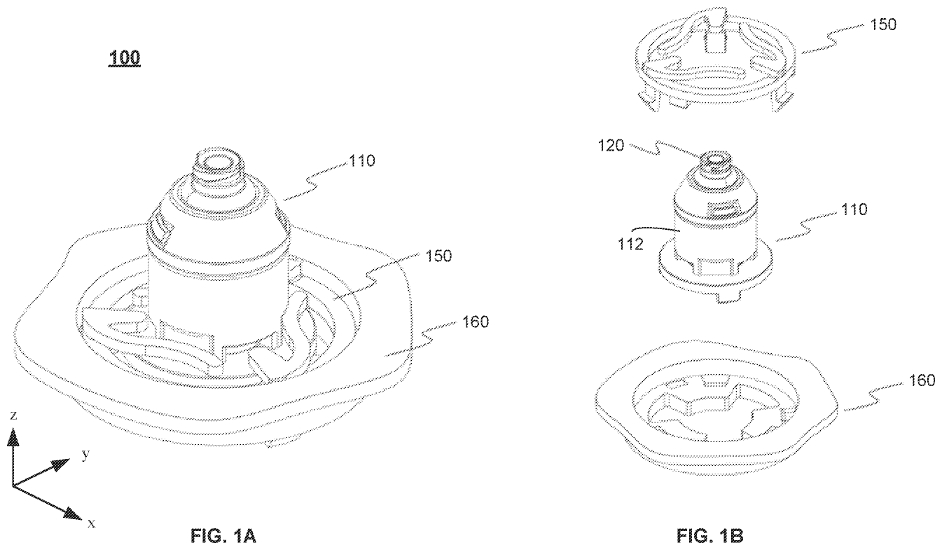

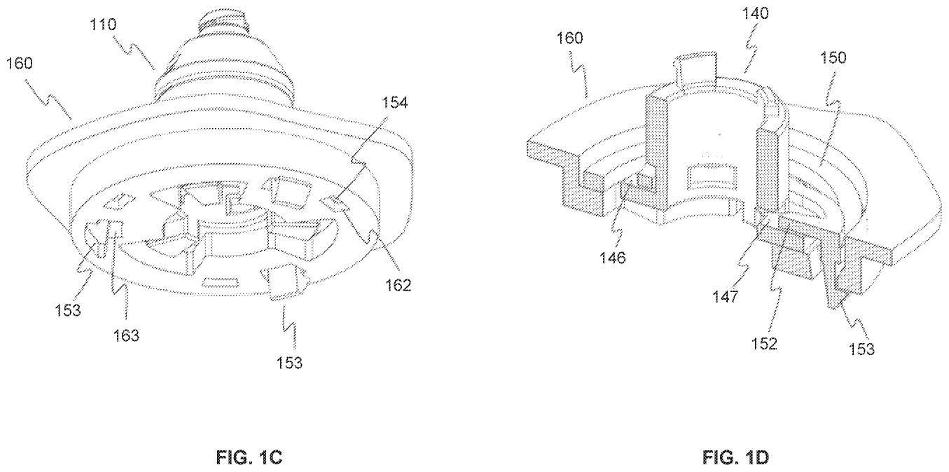

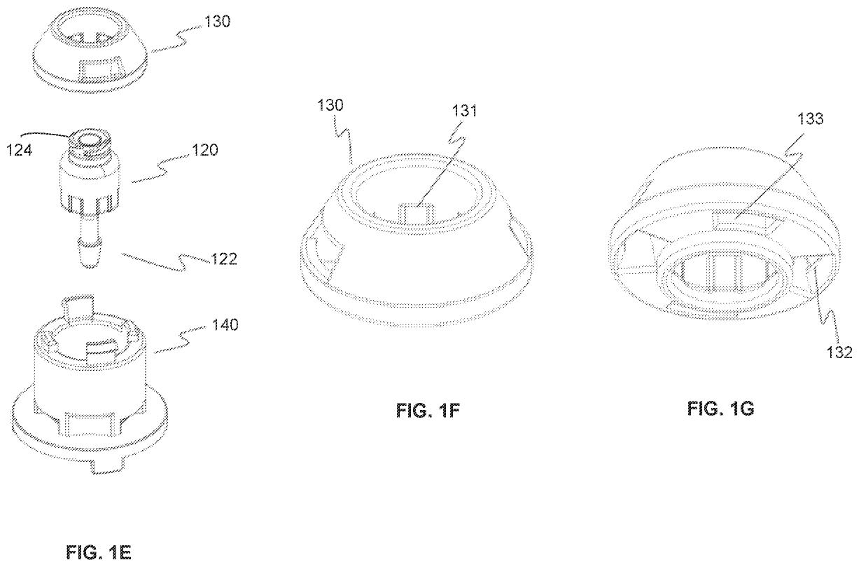

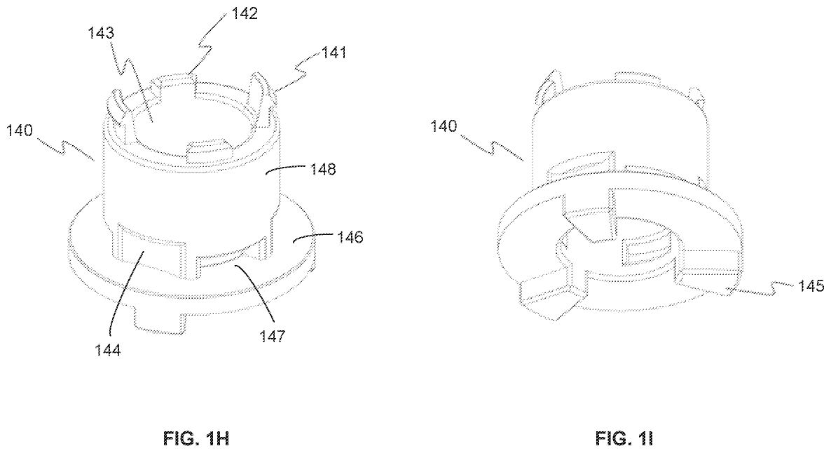

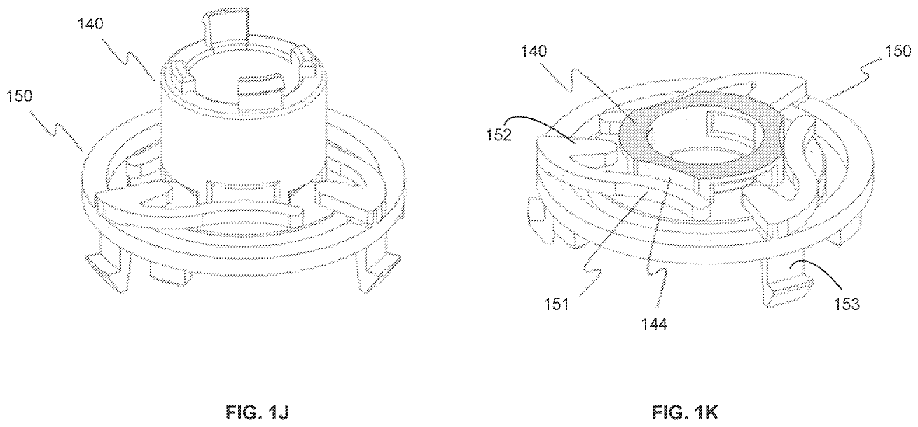

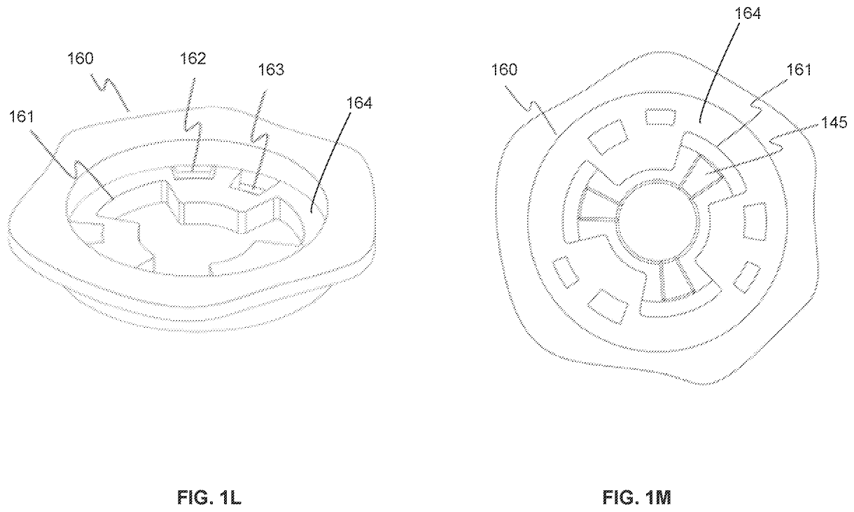

FIGS. 1A-1M illustrate an exemplary self-centering assembly 100 in accordance with some embodiments of the present disclosure. The self-centering assembly 100 generally includes a connection unit 110 and a centering unit 150, which is configured to positionally and rotationally centers the connection unit 110. In some embodiments, the connection unit 110 and the centering unit 150 are coupled with a base unit 160, which can be an integral part of any apparatus (e.g., integrally formed with the apparatus) or fixedly coupled with any apparatus (e.g., fastened, glued or mounted on a wall of the apparatus). For instance, in an embodiment, the base unit 160 is an integral part of a cartridge or other device, or fixedly coupled with a wall of a cartridge or other device. The base unit 160, and therefore the assembly 100, can be part of any device or on any surface.

When an external body (e.g., a robot arm) imposes a displacement on the connection unit 110, the centering unit 150 allows the connection unit 110 to undergo a prescribed positional and rotational displacement relative to the base unit 160, while also acting to return the connection unit to its original position as soon as the external displacement force is removed. Such an assembly can be used by a robot or operator to make connections to fluid fitting members where displacement compliance is necessary.

In some embodiments, the connection unit 110 includes a holding member 112 and a fluid fitting member 120 coupled with (e.g., supported, housed, constrained or secured by) the holding member 112. In some embodiments, the fluid fitting member 120 is fixedly coupled with the holding member, for instance, by an adhesive, a fastener, a snap-fitting or the like. In some embodiments, the fluid fitting member 120 is a fluid connector, such as a male luer-lock connector, a female luer-lock connector, a tube connector, or the like. In some embodiments, the fluid fitting member 120 includes a first end, such as a barbed end 122, for connecting a tube to the fluid fitting member. In some embodiments, the fluid fitting member 120 includes a second end, such as a lock or threaded end 124, for coupling with another fluid fitting member.

In some embodiments, the holding member 112 includes a holding piece 130 and a holding piece 140, which are coupled to each other to secure the fluid fitting member 120 in place with respect to the holding piece 130 or the holding piece 140 or both. For instance, in some embodiments, the holding piece 130 has geometric features 131 that secure the fluid fitting member 120 in place rotationally and axially. The internal geometry 143 of the holding piece 140 can also be adjusted to assist in holding the fluid fitting member 120 in place. In some embodiments, the holding piece 130 includes one or more cutouts 132, and the holding piece 140 includes one or more snaps 141 that interface with the one or more cutouts 132 to secure the connection unit 110. In some embodiments, additionally or optionally, the holding piece 130 includes one or more lug cutouts 133 and the holding piece 140 includes one or more lugs 142 that interface with the one or more lug cutouts 133 to restrain the movement of the holding piece 130 in relation to the holding piece 140, thereby preventing unnecessary forces on snaps 141. Advantageously, the holding member 112 allows the use of zip-ties or other secondary restraining mechanisms between the fluid fitting member 120 and the tube (not shown) connecting to it via, for example, the barbed end 122.

While the holding member 112 is illustrated to include two pieces coupled with each other by snap-fitting, it should be noted that this is by way of example and is non-limiting. The holding member can be made of a single piece or can be made of more than two pieces. In embodiments where the holding member is made of multiple pieces, the coupling of the pieces can be achieved by snap-fitting, or any other fastening means such as fasteners, adhesives, or interference fits. In embodiments where snap-fitting is employed, the number of cutouts, snaps and lugs can be one, two, three, four, five or more than five.

In some embodiments, the holding member 112 (e.g., the holding piece 140) includes a side wall 148 having a plurality of contoured surface areas 144 arranged circumferentially. In some embodiments, contoured surface areas in the plurality of contoured surface areas are substantially uniformly distributed in a circumferential direction, with adjacent contoured surface spaced apart from each other. In some embodiments, the contoured surface areas are symmetrically formed on the side wall. For instance, in embodiments with three contoured surface areas, the contoured surface areas are spaced 120 degrees apart. In some embodiments, the side wall 148 (e.g., the exterior surface of the side wall) is preferably cylindrical. In some embodiments, the contoured surface areas 144 are divots formed on the side wall 148. In some embodiments, each contoured surface area is a concave area on the side wall of the holding member of the connection unit.

However, the present disclosure is not limited thereto. For instance, the holding member 112 can have 2, 3, 4, 5, 6, 7, 8, 9, 10 or more than 10 contoured surface areas. In some embodiments, the holding member 112 includes at least 2, at least 3, at least 4, or at least 5 contoured surface areas. Moreover, the contoured surface areas can but do not have to be formed symmetrically or distributed uniformly. Further, a contoured surface area can also be a convex area on the side wall of the holding member of the connection unit. In addition, a contoured surface area can extend in an axial direction of the side wall 148 across any portion of the side wall. For instance, in an embodiment, a contoured surface area extends in an axial direction of the side wall across less than 50%, less than 40%, less than 30% or less than 20% of the side wall. In another embodiment, a contoured surface area extends in an axial direction of the side wall across at least 50%, at least 60%, at least 70%, at least 80% or at least 90% of the side wall.

In some embodiments, the centering unit 150 includes a plurality of contoured elastic members 151 arranged circumferentially along an axis of the centering unit (e.g., a center axis of the centering unit). In some embodiments, the contoured elastic members are symmetrically disposed at the centering unit. For instance, in embodiments with three contoured elastic members, the contoured elastic members are spaced 120 degrees apart. However, the present disclosure is not limited thereto. For instance, the centering unit 150 can have 2, 3, 4, 5, 6, 7, 8, 9, 10 or more than 10 contoured elastic members. The contoured elastic members can but do not have to be disposed symmetrically or uniformly. In addition, the number of contoured elastic members can be the same as the number of contoured surface areas or different than the number of contoured surface areas. In some embodiments, each respective contoured elastic member in the plurality of elastic members matches a corresponding contoured surface area in the plurality of contoured surface areas on the side wall of the connection unit.

The contoured elastic members 151 can be made of any suitable materials, including but not limited to plastics or metals. In some embodiments, the contoured elastic members 151 are leaf springs or cursors that ride on the surface of the side wall of the holding member 112 (e.g., the side wall of the holding piece 140) that acts as a cam mechanism. Thus, when the connection unit is subject to an external force, the cooperation of the contoured surface areas 144 and contoured elastic members 151 allows the connection unit 110 to be displaced with respect to the axis of the centering unit 150. When the external force is removed, the cooperation of the contoured surface areas 144 and contoured elastic members 151 forces the connection unit to return to its original position. For instance, when an external force that displaces the connection unit 110 is removed, the contoured elastic members 151 return to their neutral position and push the connection unit 110 back into its original axial and angular position. The contoured surface areas 144 of the side wall 148 of the holding member 112 that mate with the contoured elastic members 151 can be defined as to provide the desired centering force and centering torque in relation to the imposed deflection and thus define a radial and rotational stiffness of the system.

In some embodiments, the base unit 160 and the holding member 112 of the connection unit 110 are configured to define a maximal allowed displacement (radially, angularly or both) of the connection unit with respect to the base unit. For instance, in some embodiments, the base unit 160 includes a shouldering member 164 having one or more slots (e.g., cutouts) 161. As a non-limiting example, three slots are shown. The holding member 112 (e.g., the holding piece 140 of the holding member) of the connection unit 110 includes a flange 146 that sits on the shouldering member of the base unit. The flange 146 includes one or more protrusions (e.g., extrusions, extensions, bulges, bumps) 145 that sit in the one or more slots 161. As a non-limiting example, three protrusions are shown. As such, the one or more slots and one or more protrusions collectively serve as a hard stop, restricting the maximum rotation and maximum displacement of the connection unit 110 to the desired amount. When the one or more slots and one or more protrusions are in contact with each other, the overall assembly also reaches a much higher stiffness. In some embodiments, the hard stop features (e.g., the one or more protrusions or slots) can also present elastic elements to tune the desired end-of-travel stiffness.

While it is shown that the shouldering member 164 of the base unit 160 includes one or more slots and the holding member 112 (e.g., the flange 146) of the connection unit 110 includes one or more protrusions, it should be noted that this is by way of example and is non-limiting. The one or more slots can be formed on either the shouldering member of the base unit or the holding member of the connection unit. Similarly, the one or more protrusions can be formed on either the shouldering member of the base unit or the holding member of the connection unit. In addition, a combination of slot(s) and protrusion(s) can be formed on either the shouldering member of the base unit or the holding member of the connection unit.

In some embodiments, the centering unit 150 includes a plurality of overhangs 152, e.g., 2, 3, 4, 5, 6, 7, 8, 9, 10 or more than 10 overhangs. The number of the overhangs can the same as the number of contoured elastic members or different than the number of contoured elastic members. The overhangs are arranged circumferentially, with each overhang sitting on the flange 146 of the holding member of the connection unit. This prevents axial movement of the connection unit with respect to the centering unit or the base unit.

In some embodiments, the side wall 148 of the holding member of the connection unit further includes a plurality of slots (e.g., cutouts) 147. Each slot 147 corresponds to an overhang 152 in the plurality of overhangs, and thus provides space for the overhang when the connection unit is displaced. For instance, as the connection unit 110 is displaced in a direction, the overhang(s) 152 on one side begins to cover less of the flange, while the overhang(s) 152 on the other side begins to cover more of the flange. On the side that begins to cover more, slot(s) 147 of the connection unit 110 allows space for the overhang(s) 152. The advantage of this solution is that overhangs can be relatively large, allowing a large deflection and lower contact stresses, while keeping the overall assembly compact. The thickness and geometry of the overhangs 152 can be defined to achieve the desired vertical stiffness, acting in practice as elastic elements. In an alternative embodiment, the overhangs can have elastic elements, leaf springs or other standard elastic components, with the overhang tips fitting in the corresponding slots to achieve the desired axial stiffness.

The centering unit 150 is fixedly coupled with the base unit 160, for instance, by an adhesive, a fastener, a snap-fitting or the like. In some embodiments, the base unit 160 includes one or more slots (e.g., cutouts) 163, and the centering unit 150 includes one or more snaps 153 that interface with the one or more slots 163 of the base unit 160 to couple with the base unit. In some embodiments, the base unit 160 includes one or more slots (e.g., lug cutouts) 162, and the centering unit 150 includes one or more centering lock lugs 154 that sit in the one or more slots 162 to prevent unwanted forces on the one or more snaps 153 due to the rotational or radial displacement. However, the present disclosure is not limited thereof. For instance, in an alternative embodiments, the one or more slots are formed on the centering unit whereas the one or more snaps or lugs are formed on the base unit.

In some embodiments, the fluid fitting member 120 is held in place by the holding member 112, which is accommodated in the centering unit 150 and/or the base unit 160. In some embodiments, the geometry of the holding member matches the geometry of the centering unit and/or the base unit but allows some free translational motion (e.g., in the x-y planar direction illustrated in FIG. 1A) as well as some rotation (e.g., rotation along an axis in the z direction illustrated in FIG. 1A). In some embodiments, the slack motion is allowed in the z direction and is elastically preloaded in one direction.

In some embodiments, the fluid fitting member 120 is secured to the holding member 112 by a twisting motion of the holding member, like a luer-lock connector, and protrusions 145 on the holding member function as hard stops against the slots 161 in the base unit 160 allowing a device (e.g., a robot gripper) to exert tightening torque and thus a secure connection. In some embodiments, the hard-stop is detected by one or more sensors (e.g., sensors 1216 of FIG. 12). Examples of such a sensor include but are not limited to current sensors, electrical switches, capacitive, resistive or pressure sensors, which can be placed on a cartridge, a gripper or the like.

In some embodiments, the contoured elastic members 151 are used for keeping the holding member 112 in the same centered position. In some embodiments, the contoured elastic members are disposed symmetrically around the holding member 112. In some embodiments, the holding member 112 is also secured in the z direction by the centering unit 150 and/or the base unit 160, which secures the z-direction motion of the holding member without leaving slack or free motion. In some such embodiments, the vertical z-compliance is included in a gripper, such as any gripper disclosed herein. In some embodiments, an advantage of such arrangement is that vertical motion on the gripper is measured by a sensor (e.g., a linear encoder) to detect engagement and verify the correct engagement of the fluid fitting member, for example, in the case of a luer-lock connection, by comparing the vertical motion to the rotation of the mating receptacle.

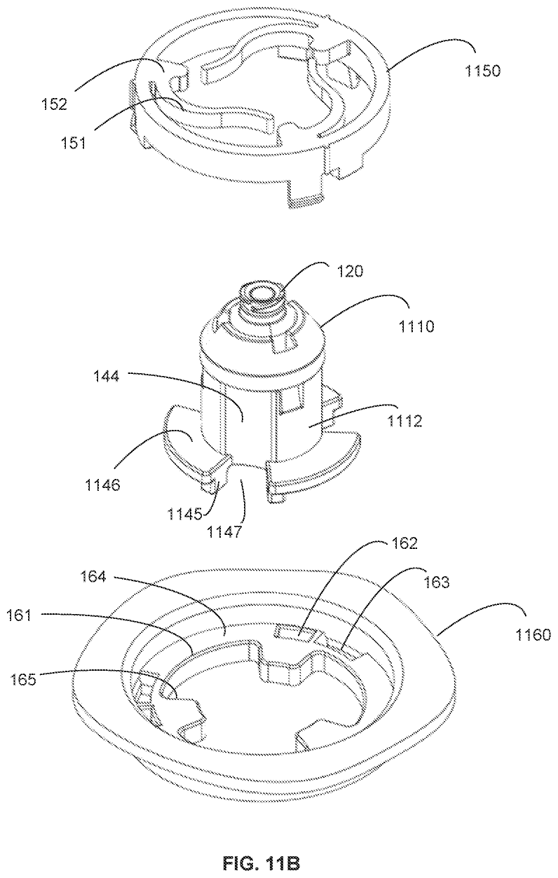

FIGS. 11A and 11B illustrate an exemplary self-centering assembly 1100 in accordance with some embodiments of the present disclosure. The self-centering assembly 1100 is similar to the self-centering assembly 100 disclosed herein. For instance, the self-centering assembly 1100 generally includes a connection unit 1110 and a centering unit 1150, which is configured to positionally and rotationally centers the connection unit 1110. In some embodiments, the connection unit 1110 and the centering unit 1150 are coupled with a base unit 1160, which can be an integral part of any apparatus (e.g., integrally formed with the apparatus) or fixedly coupled with any apparatus (e.g., fastened, glued or mounted on a wall of the apparatus). As such, the base unit 1160, and thus the assembly 1100, can be part of any part or surface.

The connection unit 1110 is similar to the connection unit 110. For instance, in some embodiments, the connection unit 1110 includes a holding member 1112 and a fluid fitting member 120 coupled with (e.g., supported, housed, constrained or secured by) the holding member 1112. The holding member 1112 includes a plurality of contoured surface areas 144 arranged circumferentially. In some embodiments, instead of a flange, the holding member 1112 of the connection unit 1110 includes a plurality of flange segments 1146. Adjacent flange segments are spaced apart from each other, forming a slot 1147 in between. A protrusion 1145 is formed at each side of a flange segment.

The centering unit 1150 is substantially the same as the centering unit 150. For instance, in some embodiments, the centering unit 1150 includes a plurality of contoured elastic members 151 and a plurality of overhangs 152. The plurality of contoured elastic members 151 and the plurality of overhangs 152 of the centering unit 1150 can be disposed at any suitable locations, e.g., on top of the side wall of the centering unit or on an interior side of the of the side wall of the centering unit. By way of example, the plurality of contoured elastic members 151 and the plurality of overhangs 152 of the centering unit 1150 are shown at the interior side of the of the side wall of the centering unit 1150. The overhangs 152 sit on the flange segments 1146 of the holding member of the connection unit. This prevents axial movement of the connection unit with respect to the centering unit or the base unit.

The base unit 1160 is substantially the same as the base unit 160. For instance, in some embodiments, the base unit 1160 includes a shouldering member 164 and one or more slots 161. The shouldering member 164 and one or more slots 161 of the base unit 1160 are coupled with the flange segments 1146, slots 1147, protrusions 1145, or a combination thereof to define a maximal allowed displacement (radially, angularly or both) of the connection unit with respect to the base unit.

The centering unit 1150 is fixedly coupled with the base unit 1160, for instance, by an adhesive, a fastener, a snap-fitting or the like. In some embodiments, the centering unit 1150 is fixedly coupled with the base unit 1160 in a similar fashion as disclosed herein with respect to the centering unit 150 and the base unit 160, e.g., through slots, snaps, and/or lugs formed on the centering unit and the base unit.

When an external body (e.g., a robot arm) imposes a displacement on the connection unit 1110, the centering unit 1150 allows the connection unit 1110 to undergo a prescribed positional and rotational displacement relative to the base unit 1160, while also acting to return the connection unit to its original position as soon as the external displacement force is removed. Such an assembly can be used by a robot or operator to make connections to fluid fitting members where displacement compliance is necessary.

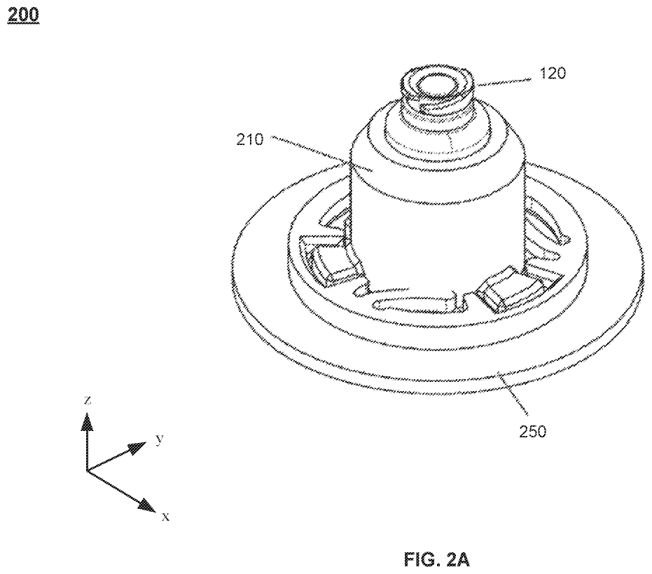

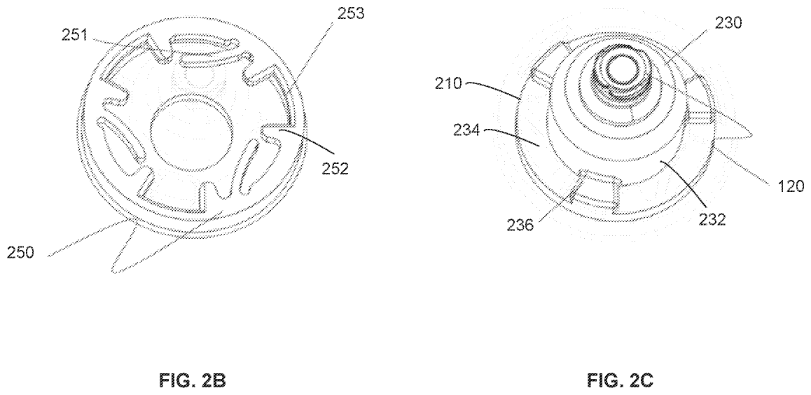

FIGS. 2A-2C illustrate an exemplary self-centering assembly 200 in accordance with some embodiments of the present disclosure. The self-centering assembly 200 generally include a connection unit 210 and a centering unit 250, which is configured to positionally and rotationally centers the connection unit 210. In some embodiments, the centering unit 250 is an integral part of any apparatus (e.g., integrally formed with the apparatus) or fixedly coupled with any apparatus (e.g., fastened, glued or mounted on a wall of the apparatus). As such, the centering unit 250, and thus the assembly 200, can be part of any part or surface.

In some embodiments, the connection unit 210 includes a holding member 230 and a fluid fitting member 120 coupled with (e.g., supported, housed, constrained or secured by) the holding member 230. In some embodiments, the fluid fitting member 120 is fixedly coupled with the holding member 230, for instance, by an adhesive, a fastener, a snap-fitting or the like. The holding member includes a cylindrical side wall 232. In some embodiments, the holding member 230 of the connection unit 210 includes a flange 234 and one or more protrusions (e.g., extrusions, extensions, bulges, bumps) 236 formed on the flange 234. As a non-limiting example, three protrusions are shown.

In some embodiments, the centering unit 250 includes a plurality of elastic members 251 arranged circumferentially along an axis of the centering unit 250. Each of the plurality of elastic members forms a contact with the cylindrical side wall 232 of the holding member 230 of the connection unit 210. This allows the connection unit to be displaced with respect to the axis of the centering unit when the connection unit is subject to an external force and forces the connection unit to return to its original position when the external force is removed. For instance, when the connection unit 210 is subject to an external force, the cooperation of the side wall of the holding member 230 and elastic members 251 allows the connection unit 210 to be displaced with respect to the axis of the centering unit 250. When the external force is removed, the cooperation of the side wall of the holding member 230 and elastic members 251 forces the connection unit 210 to return to its original position.

In some embodiments, the elastic members 251 are symmetrically disposed at the centering unit 250. For instance, in embodiments with three elastic members, the elastic members are spaced 120 degrees apart. However, the present disclosure is not limited thereto. For instance, the centering unit 250 can have 2, 3, 4, 5, 6, 7, 8, 9, 10 or more than 10 elastic members. The elastic members 251 can but do not have to be disposed symmetrically or uniformly. In addition, the elastic members 251 can but do not have to have a contour.

Like the contoured elastic members 151, the elastic members 251 can be made of any suitable materials, including but not limited to plastics or metals. In some embodiments, the elastic members 251 are leaf springs or cursors that ride on the side wall of the holding member 230 that acts as a cam mechanism.

In some embodiments, the centering unit 250 includes a plurality of overhangs 252, e.g., 2, 3, 4, 5, 6, 7, 8, 9, 10 or more than 10 overhangs. The number of the overhangs can be the same as the number of elastic members 251 or different than the number of elastic members 251. The overhangs are arranged circumferentially, with each overhang sitting on the flange 234 of the holding member of the connection unit. This prevents axial movement of the connection unit 210 with respect to the centering unit 250.

In some embodiments, the plurality of overhangs 252 collectively form one or more slots 253 to accommodate the one or more protrusions 236 of the connection unit 210. For instance, in an embodiment, an overhang 252 is disposed at each side of an elastic member 251 and adjacent overhangs form a slot 253. As such, the one or more slots 253 and one or more protrusions 236 collectively serve as a hard stop, restricting the maximum rotation and maximum displacement of the connection unit 210 to the desired amount. When the one or more slots and one or more protrusions are in contact with each other, the overall assembly also reaches a much higher stiffness. In some embodiments, the hard stop features (e.g., the one or more protrusions or slots) can also present elastic elements to tune the desired end-of-travel stiffness.

While it is shown that the centering unit 250 includes one or more slots and the holding member 230 of the connection unit 210 includes one or more protrusions, it should be noted that this is by way of example and is non-limiting. The one or more slots can be formed on either the centering unit 250 or the holding member of the connection unit 210. Similarly, the one or more protrusions can be formed on either the centering unit 250 or the holding member of the connection unit 210. In addition, a combination of slot(s) and protrusion(s) can be formed on either the centering unit 250 or the holding member of the connection unit 210.

In some embodiments, the fluid fitting member 120 is held in place by the holding member 230, which is accommodated in the centering unit 250. In some embodiments, the geometry of the holding member matches the geometry of the centering unit but allows some free translational motion (e.g., in the x-y planar direction illustrated in FIG. 2A) as well as some rotation (e.g., rotation along an axis in the z direction illustrated in FIG. 2A). In some embodiments, the slack motion is allowed in the z direction and is elastically preloaded in one direction. In some embodiments, the fluid fitting member 120 is secured to the holding member 230 by a twisting motion of the holding member, like a luer-lock connector, and protrusions 236 on the holding member function as hard stops against the slots 253 in the centering unit 250 allowing a device (e.g., a robot gripper) to exert tightening torque and thus a secure connection. In some embodiments, the hard-stop is detected by sensors (e.g., sensors 1216 of FIG. 12). Examples of such a sensor include but are not limited to current sensors, electrical switches, capacitive, resistive or pressure sensors, which can be placed on a cartridge, a gripper or the like.

In some embodiments, the elastic members 251 are used for keeping the holding member 230 in the same centered position. In some embodiments, the elastic members are disposed symmetrically around the holding member 230. In some embodiments, the holding member 230 is also secured in the z direction by the centering unit 250, which secures the z-direction motion of the holding member without leaving slack or free motion. In some such embodiments, the vertical z-compliance is included in a gripper, such as a gripper disclosed herein. In some embodiments, an advantage of such arrangement is that vertical motion on the gripper is measured by a sensor (e.g., a linear encoder) to detect engagement and verify the correct engagement of the fluid fitting member, for example, in the case of a luer-lock connection, by comparing the vertical motion to the rotation of the mating receptacle.

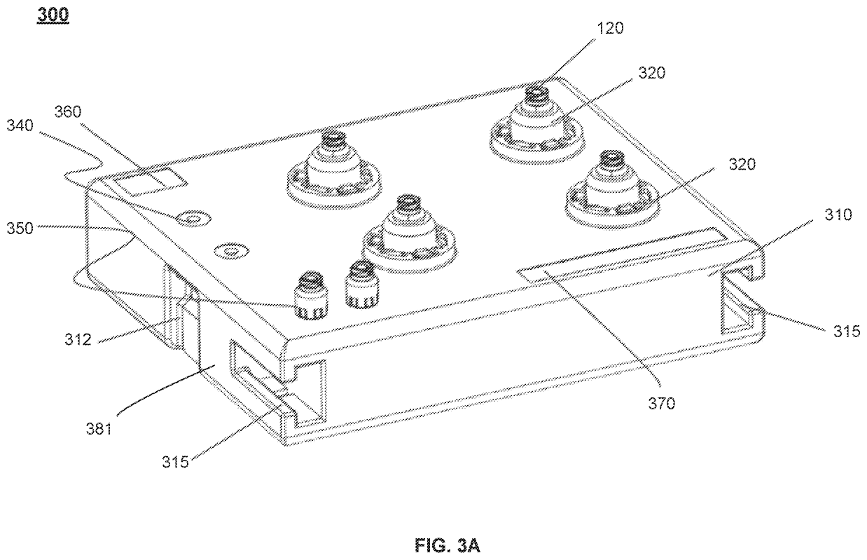

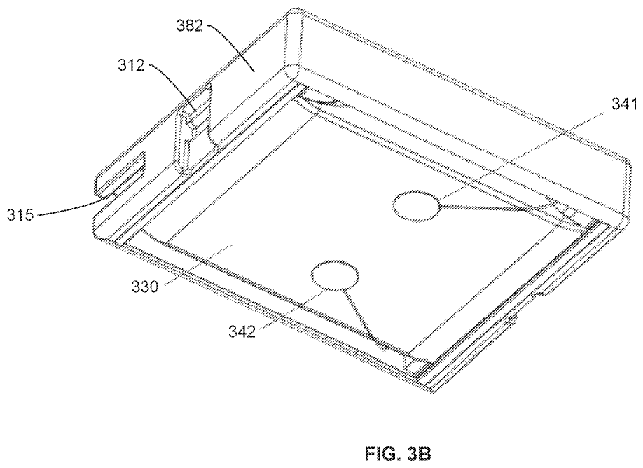

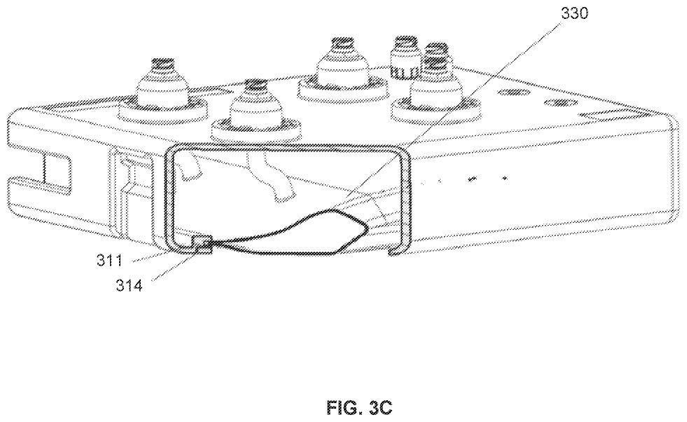

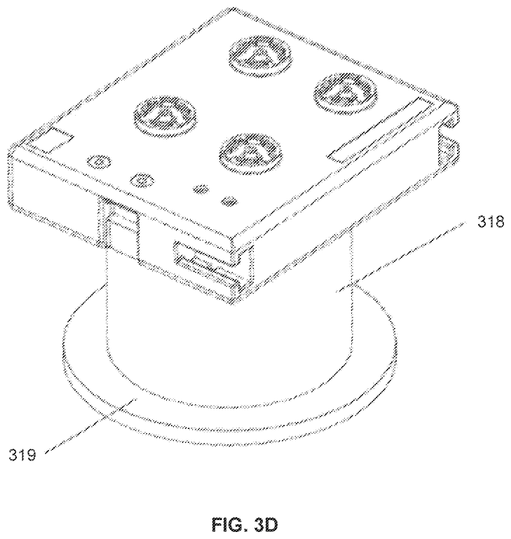

FIGS. 3A-3B illustrate an exemplary cartridge 300 in accordance with some embodiments of the present disclosure. The cartridge 300 can be moved, for instance, by a robotic arm, and placed in a dedicated equipment (e.g., instrument). Examples of such an equipment include but are not limited to a thawing machine, a freezer, a bioreactor, a fill and finish machine, or other equipment with specific functions for manufacture of a capsule and/or cell therapy.

The cartridge 300 generally includes a cartridge body 310 and one or more connectors 320 coupled with the cartridge body (e.g., mounted on a wall of the cartridge body). The cartridge 300 can include any suitable number of connectors 320. For instance, in an embodiment, the cartridge 300 consists of a single connector 320. In an alternative embodiment, the cartridge 300 includes 2, 3, 4, 5, 6, 7, 8, 9, 10, or more than 10 connectors 320. In some embodiments, a connector 320 is a self-centering assembly, such as the self-centering assembly 100, 200 or 1100 disclosed herein. In some embodiments, the holding member of the self-centering assembly is attached to the cartridge after the holding member is located by standard means such as bonding, thermal welding, snap features or fasteners.

In some embodiments, a fluid (e.g., gas, liquid, solution, etc.) is transferred to and from the cartridge 300 or one or more containers connected to the cartridge through the one or more connectors 320, which are allowed to move relative to the cartridge body 310 by a small amount or motion slack. In some embodiments, this amount is the tolerance that provides manufacturing tolerances, positioning tolerances, as well as robot repeatability errors. In some embodiments, the fluid fitting member 120 of the connector can be located and/or pushed by a gripper with aligning and centering features, such as conical surfaces, ramps, bearing surfaces.

In some embodiments, the cartridge 300 includes one or more locking members for transporting the cartridge and/or placing the cartridge in a dedicated equipment. For instance, in some embodiments, the cartridge includes one or more first locking members 312 and one or more second locking members 315. The first and second locking members are generally orthogonal to each other. This allows the use of passive elements for locking and supporting the cartridge 300 in either a gripper or an equipment, e.g., providing retention force when the cartridge snaps in place with the gripper or equipment. Examples of such a locking member include but are not limited to cantilevered snaps, spring loaded protrusions, slots, locating pins, ramps or bearing surfaces.

In some embodiments, the one or more first locking members 312 and/or the one or more second locking members 315 engage (e.g., mate) with one or more matching members on a gripper or equipment to create retention force. For instance, in some embodiments, the one or more first locking members 312 and/or the one or more second locking members 315 engage (e.g., mate) with one or more matching members on a gripper, allowing the gripper to self-center the cartridge and to safely transport and load the cartridge into an equipment, storage, or disposal. In some embodiments, the one or more first locking members 312 and/or the one or more second locking members 315 guide and self-center the cartridge 300 in the equipment while at the same time providing support against operational loads acting in different directions, for instance, due to fluid moving in and out of the cartridge 300. In some embodiments, the cartridge is retained in place by motorized jaws or fingers of a gripper or equipment.

In some embodiments, the one or more first locking members 312 and the one or more second locking members 315 are formed on the cartridge body 310. In some embodiments, the cartridge body 310 is a relatively rigid shell. In an embodiment, a first locking member 312 and a second locking member 315 are formed on a first side 381 of the cartridge body 310 and a first locking member 312 and a second locking member 315 are formed on a second side 382 of the cartridge body 310. The first side 381 and second side 382 are opposite to each other. In some embodiments, a first locking member 312 includes a cantilevered snap or a spring loaded protrusion while a second locking member 315, orthogonal to the first locking member 312, includes a slot.

In some embodiments, the cartridge 300 include one or more optional, additional or alternative components. For instance, in some embodiments, the cartridge 300 includes one or more electronics connectors 340, one or more sensors or sensor pads 341, 342, one or more additional fluid connectors 350, a reference tag 360, a bar code tag 370, or any combination thereof. In some embodiments, the electronics connectors 340 allow the cartridge to be electrically connected (e.g., by a robot or an operator) to an equipment. This provides power for active systems(s), component(s) such as sensors, process(es) such as temperature control, communication(s) such as data exchange, or other devices or processes. In some embodiments, the one or more additional fluid connectors 350 are used for manual operations or inspections. In some embodiments, the fluid connector 350 is a needle free luer lock connector. In some embodiments, the reference tag 360 is used by a robot to register, for instance, via a camera mounted on the robot arm, a gripper's location relative to the cartridge. In some embodiments, the bar code tag 370 is used to provide specific information, such as the specific information about the cartridge, the fluid to be transferred, or the like.

In some embodiments, the cartridge 300 is configured to secure one or more fluid containers, which can be used to collect a fluid transferred through the connectors. A fluid container can be flexible or rigid. For instance, as a non-limiting example, FIG. 3C illustrates a flexible container (e.g., a bag) 330 secured in the cartridge 300. In some embodiments, the flexible container is secured inside the cartridge body 310 of the cartridge 300. As another non-limiting example, FIG. 3D illustrates a rigid container (e.g., a flask) 318 connected to the cartridge 300. In some embodiments, a membrane 319 is placed on a surface of the container (e.g., at the bottom of it) to allow specific exchanges between the contained fluid and the outer atmosphere. For instance, in an embodiment, the membrane 319 allows the contained fluid (e.g., cells) to exchange oxygen with the outer atmosphere (e.g., air).

In some embodiments, the cartridge 300 includes one or more retaining members 311 to retain a container (e.g., a bag) in place. In some embodiments, the cartridge 300 includes one or more locking tabs 314 to allow a robot or an operator to lock the container on the cartridge during assembly or disassembly. In some embodiments, the cartridge body 310 is configured such that a portion of it (e.g., the bottom of the cartridge) can be opened to allow proper functioning of the equipment, for example, to allow heat exchange with the bag 330 or contact with the sensors or sensors pad 341, 342.

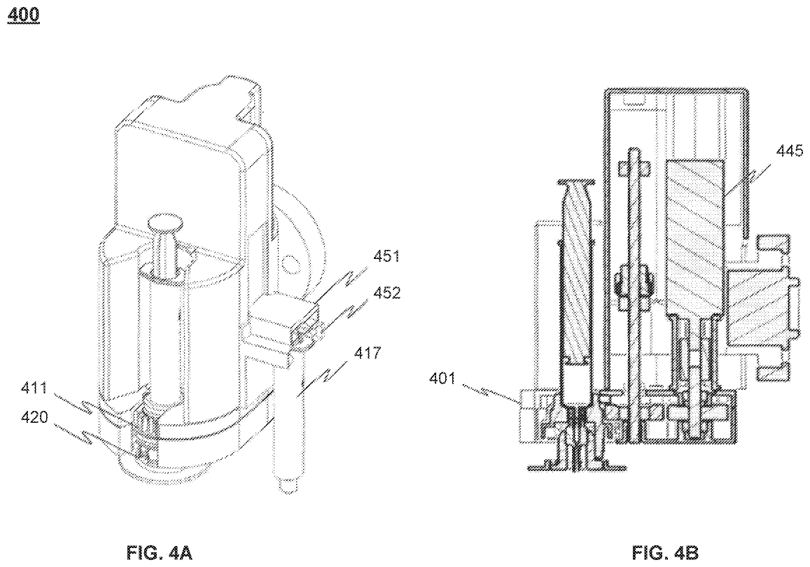

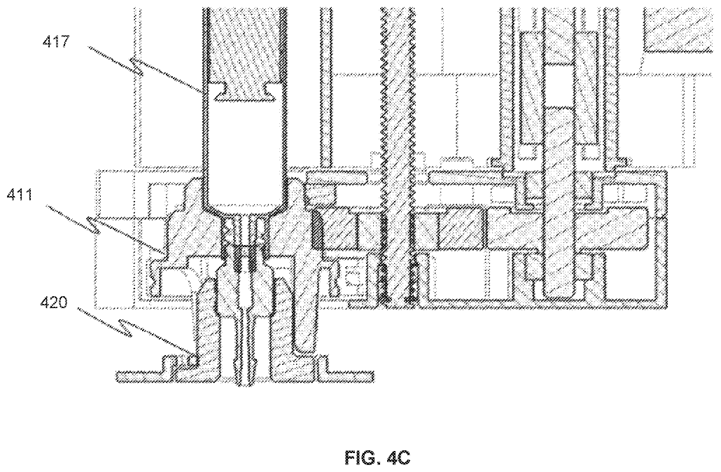

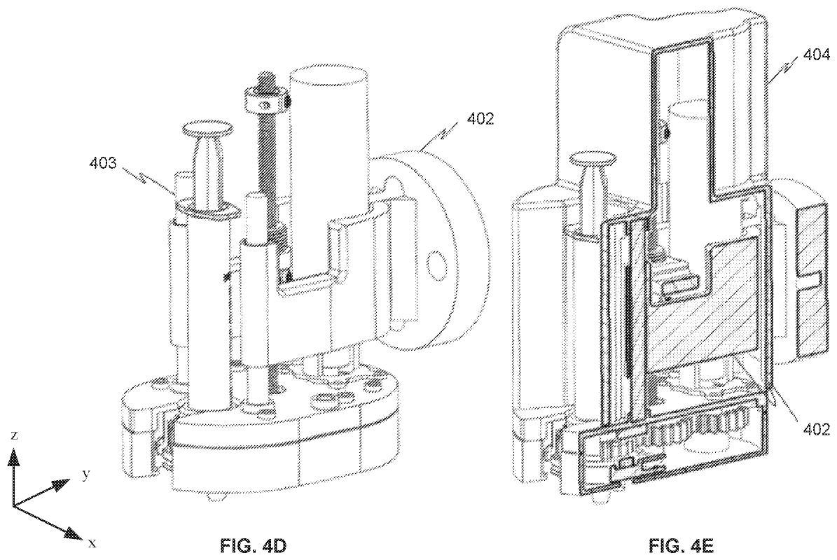

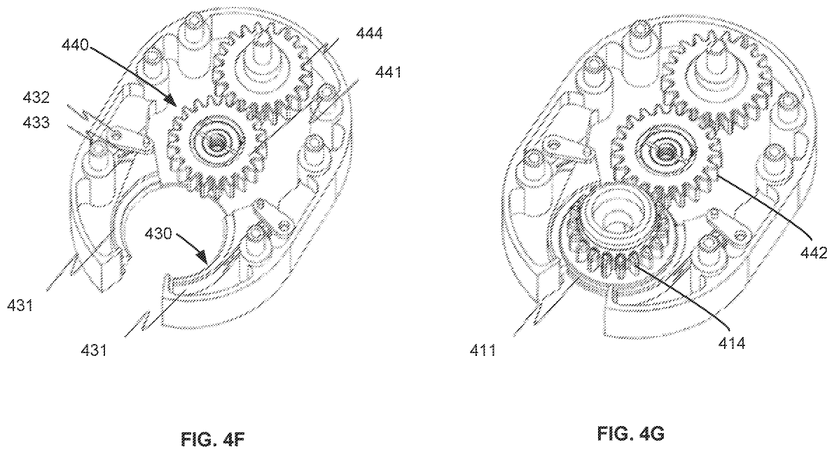

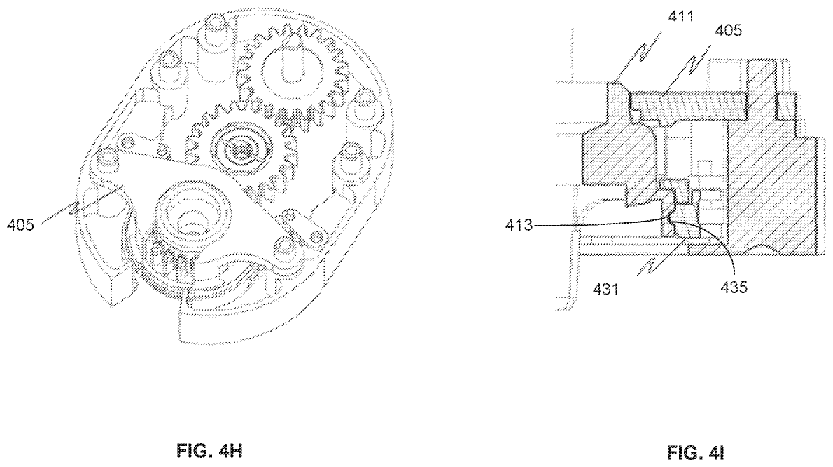

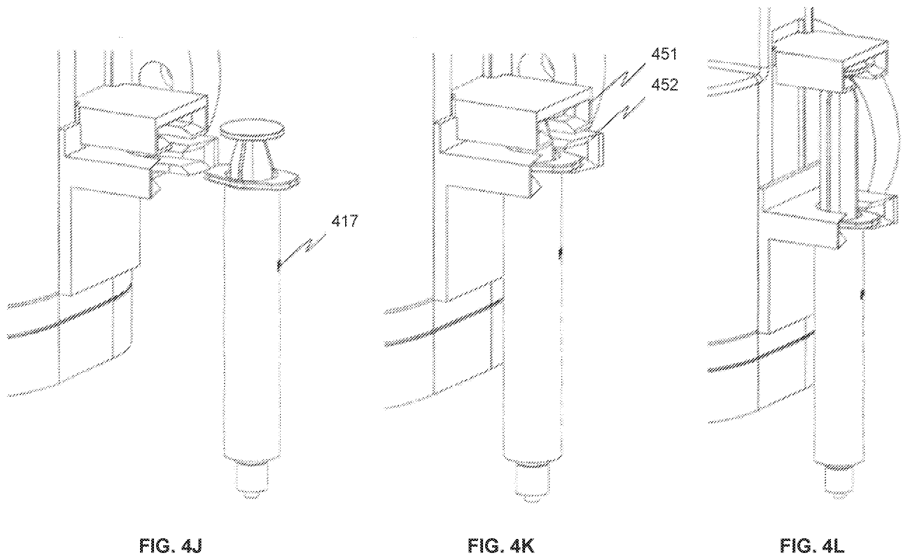

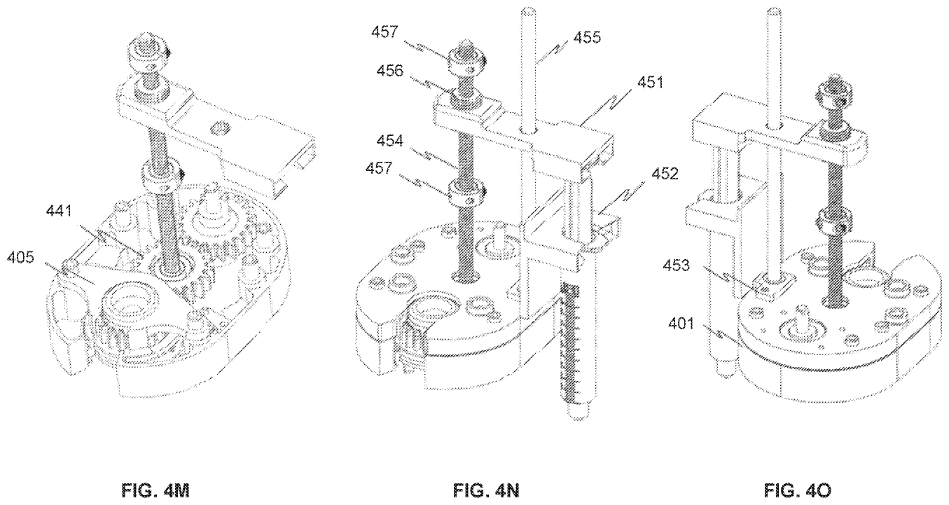

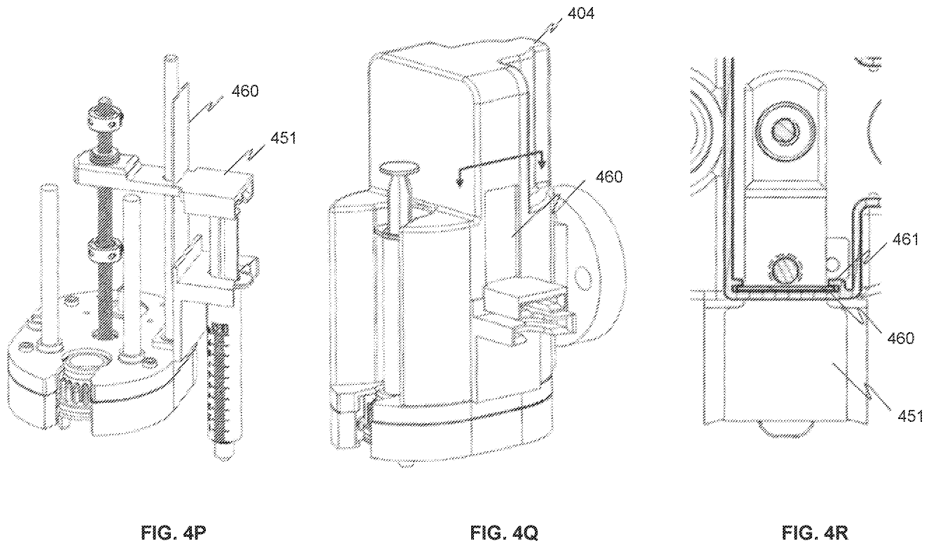

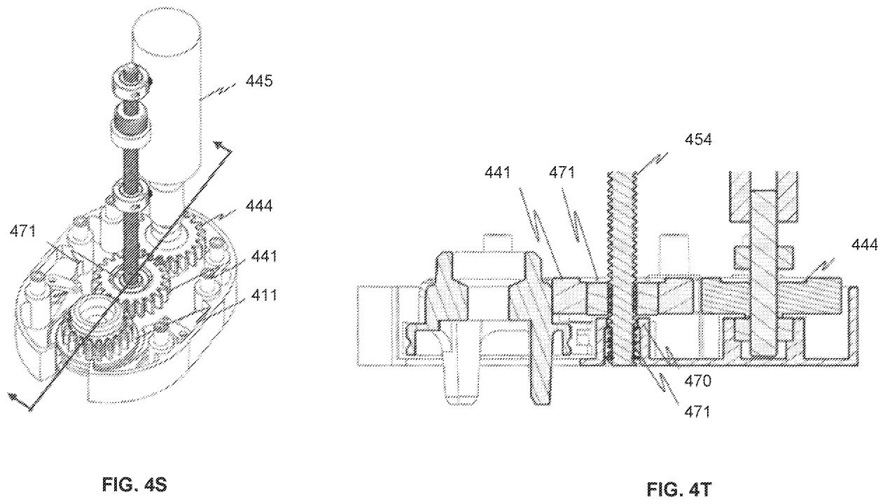

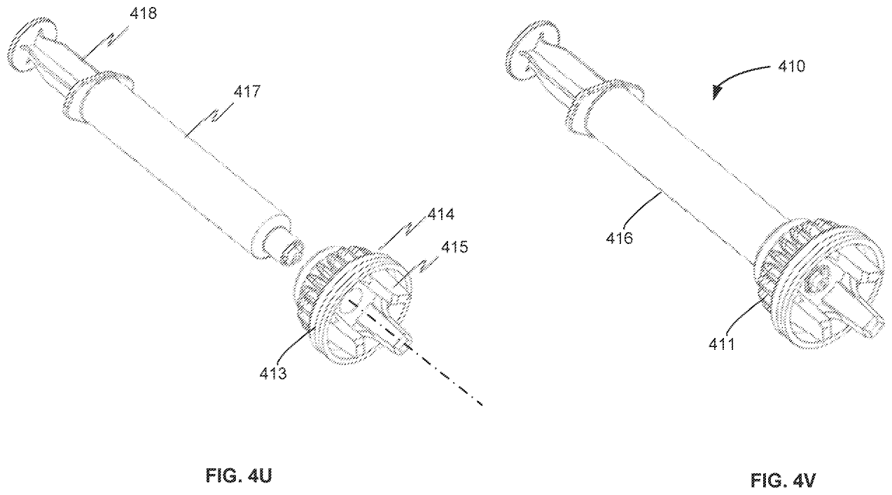

In some embodiments, once the cartridge is loaded onto a nest assembly, the connection is made with any of the connectors 320. In some embodiments, the connector 320 includes one end of an air-tight connector, such as a female Luer connector, which when mated with a male Luer connector produces an air-tight connection. In some embodiments, the connector 320 consists of one end of an air-tight connector. In some embodiments, the male Luer connector is screwed on to the female luer connector, such as via a rotating tool, such as any gripper disclosed herein, with a compatible luer connector adapter (e.g., the holding member 411 of FIG. 4V). Additionally, in some embodiments, to have repeatable and reliable connections, one or more locating features such as chamfered extrusions 415 from the holding member is used, as well as features on the gripper to enable visual recognition with a camera and/or depth sensors, edge finders, contact sensors, or a combination thereof.

FIGS. 4A-4T illustrate an exemplary gripper 400 in accordance with some embodiments of the present disclosure. The gripper 400 is configured to connect and disconnect connectors, tubes, syringes or other devices or components that require twisting of a locking element for a successful fluid connection, to grip and transport one or more connectors, tubes, syringes or other devices or components to different ports of connectors (e.g., self-centering assemblies disclosed herein), to draw and inject fluid through the connections, or a combination thereof. The gripper 400 can be operated by a robotic arm or other versatile manufacturing equipment.

The gripper 400 generally includes a gripping unit 430 and a gear unit 440. In some embodiments, the gripper 400 is configured to connect a first unit 410 with a second unit 420 or disconnecting the first unit from the second unit. The second unit can be any connector or fluid fitting device or component. For instance, in some embodiments, the second unit is a connection unit of a self-centering assembly, such as the self-centering assembly 100, 200 or 1100 disclosed herein. In a typical application, the second unit is an integral part of an equipment (e.g., a cartridge) or is secured on an equipment or a surface.

In some embodiments, the first unit 410 includes a first holding member, such as a holding member 411 illustrated in FIGS. 4U and 4V. The first holding member has a first rotation axis, illustrated by the dotted dash line in FIG. 4U. In some embodiments, the first holding member includes an engaging member, such as an engaging member 413. In some exemplary embodiments, the engaging member 413 is a groove or a protrusion on a side wall of the first holding member around the first rotation axis. In some embodiments, the first holding member includes a tooth pattern, such as a first tooth pattern 414, on a side wall of the first holding member.

In some embodiments, the first holding member 411 includes one or more locating members, such as the locating members 415 at a side facing the second unit when the first unit is brought toward the second unit and configured to help locating the first holding member of the first unit with respect to the second unit. In some embodiments, a locating member is configured in a form of an extrusion extending along the rotation axis. In some embodiments, the first holding member includes a plurality of extrusions, e.g., 2, 3, 4, 5, 6, 7, 8, 9, 10 or more than 10 extrusions, which are disposed circumferentially. As a non-limiting example, three extrusions are illustrated. The plurality of extrusions helps centering the first holding member with respect to the second unit. In some embodiments, a locating member 415 is an edge finder or chamfered extrusion that matches with a holding member of the second unit such as a holding member 112 or 230 of any self-centering assembly disclosed herein.

In some embodiments, the first unit 410 includes a first fitting member, such as a first fitting member 416, fixedly connected to the first holding member. While FIGS. 4U and 4V illustrates the first fitting member 416 in a form of a syringe, it should be noted that this is by way of example and is non-limiting. For instance, in some embodiments, the first fitting member 416 is a tube or a tube fitting member, a connector (e.g., a female or male luer lock connector), a vessel (e.g., a fluid container in a shape other than a syringe), or the like. In some embodiments, a syringe includes a syringe body 417 and a plunger 418 movable relative to each other to draw or inject a material (e.g., fluid).

In some embodiments, the first fitting member 416 (e.g., the syringe) is housed within and/or fixed to the first holding member 411, for instance, by standard means such as adhesive materials (e.g., one or more strips of adhesive), bonding, thermal welding, or mechanical means (e.g., congruent meshing features). Thus, when the first holding member rotates, the locking feature of the first holding member (e.g., the syringe tip) rotates along with the first holding member. In some embodiments, this locking mechanism applies to various modes of fluid transfer, such as tube connectors or self-centering assemblies disclosed herein.

In some embodiments, the gripping unit 430 of the gripper 400 is operable to engage with the first holding member 411 of the first unit 410. When the gripping unit is engaged with the first holding member of the first unit, the gripping unit restricts the first holding member of the first unit from moving axially with respect to the gripping unit (e.g., the z-direction illustrated in FIG. 4D) but allows the first holding member of the first unit to rotate around the first rotation axis of the first holding member.

For instance, referring to FIG. 4I, in some embodiments, the gripping unit 440 includes a second engaging member, such as a second engaging member 435 at its interior side that matches the first engaging member 413 of the first holding member of the first unit. While FIG. 4I illustrates that the first engaging member 413 is a groove and the second engaging member 435 is a protrusion that fits with the groove, it should be noted that this is by way of example and is non-limiting. For instance, in some embodiments, the second engaging member 435 is a groove whereas the first engaging member 413 is a protrusion that fits with the groove. In some embodiments, the first and/or second engaging members are of a different shape, like wedges, ramps, snaps, cylinders or others that provide mechanical engagement. In some embodiments, the first and/or second engaging members include additional or alternative or optional mechanism or material whose attractive force is related to proximity of another part, such as a magnet. The coupling of the first engaging member of the first unit and the second engaging member of the gripping unit allows the first holding member to rotate relative to the gripping unit while securing (e.g., limiting or restricting) the axial motion of the first holding member relative to the gripping unit.

In some embodiments, the gripping unit 430 includes one or more gripping members, such as the gripping member(s) 431. Each of the one or more gripping members is rotatable around an axis parallel to the rotation axis of the first holding member of the first unit. For instance, in some embodiments, each gripping member 432 is rotatable around a pin 432 as illustrated in FIG. 4F. In some embodiments, the pin 432 is concentric with a torsional spring 433 that provides elasticity to the gripping member about this pin. In some embodiments, the one or more gripping members are made from an elastic material, are cam mechanism, are linkage mechanism (e.g., 4-bar mechanism), preloaded linear springs, or a combination thereof, that is configured to provide the one or more gripping members a tendency to move inwardly (e.g., towards the first holding member 411 when to grip the first unit 410).

The gripping unit 430 can include any suitable number of gripping members, e.g., 1, 2, 3, 4, 5, or more than 5 gripping members. In some embodiments, the gripping unit 430 includes two gripping members symmetrical relative to each other, and/or relative to the axis of rotation of the first holding member. In some embodiments, when the gripping unit is engaged with the first holding member of the first unit, the gripping member defines a gripping axis that coincides with the rotation axis of the first holding member of the first unit.

In some embodiments, the gear unit 440 of the gripper 400 includes a first gear 441 operable to rotate the first holding member 411 of the first unit 413 around the first rotation axis. For instance, in some embodiments, the gear unit 440 includes a second tooth pattern 442 that matches the first tooth pattern 414 of the first holding member 411. The first gear 441 can be driven by a motor or another gear (e.g., a second gear 444 illustrated in FIG. 4F).

In some embodiments, when the first unit 410 or the first fitting member 416 (e.g., the syringe) of the first unit 410 is to be unscrewed from the second unit 420 (e.g., a female luer lock), the gripper 400 is first lowered onto the first holding member 411 so that the second engaging member 435 of the gripping unit 430 is coincident with the first engaging member 413 on the first holding member 411. In some embodiments, the one or more locating members 415 of the first holding member 411 provide self-centering features and planar compliance, and thus help to secure the first unit 410 unto a corresponding second unit 420 or a fluid fitting member (e.g., a female luer lock connector) of the second unit successfully.

In some embodiments, as the gripper 400 is lowered onto the first holding member 411, the rounded or chamfered profile of the first holding member interferes with the gripping unit 430 and pushes the gripping unit open. In some embodiments, once the gripping unit 430 (and in some cases the gripper 400) is lowered to a certain location relative to the first holding member, the gripping unit snap inward and onto the first holding member and grip it with the gripping unit, e.g., with the convex groove of gripping unit that is congruent with the concave groove of the first holding member as illustrated in FIG. 4I. In some embodiments, these congruent grooves are of a different shape, like wedges, ramps, snaps, cylinders or others that provide mechanical engagement. In some embodiments, the mating of these grooves or features secures the first holding member axially relative to the gripping unit, but allows for rotation of the first holding member relative to the gripping unit. In some embodiments, the mating of these grooves or features includes a mechanism or material whose attractive force is related to proximity of another part, such as a magnet.

In some embodiments, once the one or more gripping members of the gripping unit 430 are located against the first holding member, the first gear (e.g., lead screw gear) 441 rotates, for instance, driven by the second gear (e.g., motor gear) 444 and/or the motor 445, whose rotational axis is connected to the second gear 444 to transmit torque. In some embodiments, the first holding member, first gear, second gear, or a combination thereof have involute tooth patterns so that power is transmitted therebetween. In some embodiments, power is transmitted through a different tooth pattern or frictional surface. As the second gear rotates, the second gear rotates the first holding member 411, and thus rotates the fitting member (e.g., syringe, male luer lock, etc.) 416 housed and fixed to the first holding member. The rotation unscrews the first holding member 411 and the fitting member 416 from the second unit 420 or the fitting member of the second unit (e.g., female luer lock).

In some embodiments, the gripper 400 includes a capping member, such as a capping member 405 illustrated in FIGS. 4H and 4I. The capping member is configured to constrain translation movement of the first holding member in a plane normal to the rotation axis of the first holding member, translation movement of the first holding member along the rotation axis of the first holding member, or both. In some embodiments, the capping member serves as a hard stop to define a boundary to prevent the gripping unit from closing further inwardly. For instance, as the second gear rotates the first holding member, tangential and axial forces transmitted between the first holding member and the first gear are compensated by the capping member 405, which constrains the first holding member against both translation along the plane normal to the axis of rotation as well as axial translation, but allowing rotation along the rotation axis of the first holding member. In some embodiments, the capping member includes surfaces congruent to those of the first holding member above and/or below the gear's point of contact, and thus acts as a dual bearing surface for the rotation of the first holding member. This structure also allows the capping member to secure the first holding member, e.g., allowing its rotation along its axis while reacting to off-axis moments as well as rotation forces. In some embodiments, the capping member is configured as a hard-stop (e.g., arrest) for the gripping unit to prevent the gripping unit from closing inwardly to such a degree that would create a malfunction upon the gripper lowering onto the first holding member. However, the present disclosure is not limited thereto.

When the first holding member of the first unit rotates around the first rotation axis, the first holding member of the first unit, the gripping unit and the first gear of the gearing unit move axially with respect to the second unit toward or away from the second unit, thereby connecting the first unit with the second unit or disconnecting the first unit from the second unit. For instance, in some embodiments, the gripper 400 includes a housing member 401 configured to accommodate the gripping unit, capping member, first gear, second gear, motor, or a combination thereof. In some embodiments, the gripper 400 includes a mounting member 402 for interfacing with an automated equipment (e.g., a robot) so that a robot or other automated equipment can be coupled with the gripper 400 through the mounting member 402. In some embodiments, the mounting member 402 is a spring bracket. In some embodiments, the mounting member 402 has one or more features that not only allow it to be fastened securely to the end effector of a robot or some other automated equipment but also house the electronics to control the encoders and sensors of the gripper 400.

The mounting member 402 and the housing member 401 are movable relative to each other in the z-direction as illustrated in FIG. 4D, which is the same as the axial direction of the first holding member 411 when the first holding member is gripped by the gripper. For instance, in some embodiments, the gripper 400 includes one or more shafts 403, each fixedly connected to the housing member 401 and extending along the z-direction. The mounting member 402 is coupled to the one or more shafts 403 and slidable along the one or more shafts 403. The gripper can include any suitable number of shafts. As a non-limiting example, two shafts 403 are shown.

As the fitting member 416 (e.g., syringe) of the first unit 410 travels axially away from the second unit (e.g., female luer lock) 420 during unscrewing, the first holding member 411 of the first unit 410 and the housing member 401 (e.g., including the gripping unit, capping member, first gear, second gear, motor, or a combination thereof) of the gripper 400 move with the fitting member 416 of the first unit 410. In some embodiments, this entire aforementioned structure has axial compliance from the mounting member 402 that rides axially along the one or more shafts 403 that are fixed to the housing member 401 and/or covering member 404. In some embodiments, one or more linear springs are provided, whose ends are constrained by the stacking of the housing member, the mounting member, and the covering member, each concentric with a corresponding shaft in the one or more shafts. In some embodiments, during normal operational loads, the one or more linear springs are constantly preloaded and allow the gripper its axial compliance.

In some embodiments, the gripper 400 includes a covering member 404 to protect some components of the gripper, for instance, to enclose one or more moving parts of the gripper (e.g., the motor), making the gripper easy to clean and resistant to water ingress. In some embodiments, the one or more shafts are additionally or optionally connected to the covering member. In some embodiments, the mounting member is allowed to move axially by a linkage mechanism (e.g., a 4-bar mechanism), a cam mechanism or other mechanisms based on rotating elements instead of linear slides.

In some embodiments, the axial movement of the first holding member 411 of the first unit 410, the gripping unit 430, the first gear 441 of the gearing unit, the housing member 401, the mounting member 402 with respect to the second unit (e.g., toward or away from the second unit) is detected to ensure proper connection or disconnection. For instance, in some embodiments, one or more sensors (e.g., sensors 1216 of FIG. 12) are configured to detect this vertical motion and correlate the vertical motion to angular rotation of the motor to validate the correct engagement or disengagement of the first and second units (e.g., engagement or disengagement of luer-lock threads). Examples of such a sensor include but are not limited to linear encoders, contactless distance sensors, or the like. In some embodiments, the one or more sensors are located in the gripper. In some embodiments, the one or more sensors provide a safety feature for ensuring a correct connection before a fluid is exchanged through the connection formed by the first and second units.

In some embodiments, the one or more shafts 403 are located proximate (e.g., as close as possible) to the rotation axis of the first holding member 411 of the first unit 410 to limit the moment loads between the one or more shafts and the mounting member. In some embodiments, this limiting of the moment loads helps maintain the resistance to vertical passive motion to a minimum so the system (e.g., the computer system 12) is capable of detecting small vertical forces when using a respective sensor to measure a deflection. In some embodiments, the limiting of the moment loads is provided by utilizing an elastic material that is also guided, such as by a rail or some other component that allows for axial vertical travel yet compensates for off-axis moments.

In some embodiments, the gripper 400 includes a draw/injection unit, such as a draw/injection unit 450 illustrated in FIGS. 4A and 4J-4R, configured to draw or inject a material (e.g., a fluid) into or from a syringe (e.g., the first fitting member 416 of the first unit 410 in the form of a syringe or other syringes). The draw/injection unit 450 generally includes a carriage member 451 and a body gripping member 452 configured to interface with the plunger 418 and body 417 of the syringe, respectively. In some embodiments, this interface with the plunger and the body is provided by utilizing one or more mating features congruent with the syringe's geometry. The mating features allow the syringe to be inserted in any rotational orientation using one or more compliance features such as, one or more chamfers, one or more spring-actuated claws, one or more clearances, or a combination thereof. In some such embodiment, the carriage member and body gripping member interface through one or more bearing surfaces that allow vertical forces (e.g., along the displacement axis of the plunger) to be transmitted between the carriage member and body gripping member. In some such embodiments, the interface of the carriage member and body gripping member is space efficient as the gripper 400 is held in place through spatial, geometric constraints (e.g. as opposed to power driven ones).

In some embodiments, the body gripping member 452 is fixedly connected to the housing member 401 by a fastening member 453, for instance, by one or more fasteners, one or more brackets, snap-fitting, one or more adhesive materials (e.g., one or more strips of adhesive) or other mechanism.

In some embodiments, the draw/injection unit 450 includes an actuating member 455 to move the carriage member 451 relative to the body gripping member 452. In some embodiments, the actuating member is a screw driven by the first gear 441 of the gear unit 440. The carriage member 451 is connected to a nut 456 on the screw, for instance, by fastening, bonding, welding, or any other suitable mechanism, so that the carriage member moves together with the nut on the screw. However, the present disclosure is not limited thereto. Other mechanisms can be used to actuate the carriage member 451.

In some embodiments, the actuating member (e.g., screw) 454 to the housing member 401 is provided by using a plurality of translational constraints. In some such embodiments, the actuating member (e.g., screw) 454 is kept upright as well as constrained translationally by a plurality of bearings 471 housed in the bearing housing 470 and held up (e.g., coupled) with one or more mechanical fasteners. In some embodiments, the constraint of movement of the actuating member to the housing member 401 is at various locations using one or more bearings, one or more bushings, one or more fasteners, or a combination thereof for free rotary movement.

In some embodiments, the motor 445 drives the actuating member (e.g., screw) 454 through the second gear 444 and the first gear 441 that is fastened to the actuating member through one or more mechanical fasteners, such as one or more press-fits, one or more adhesive materials (e.g., one or more strips of adhesive), a congruent meshing, one or more set-screws, one or more clamps, bonding, or a combination thereof.

In some embodiments, one or more hard stops 457 are used to define lowest and/or highest points that the carriage member can travel. Examples of such a hard stop include but are not limited to mechanical fasteners, limit switches, sensors, magnets, or a combination thereof. As a non-limiting example, FIG. 4N illustrates two mechanical fasteners disposed on the screw.

In some embodiments, the draw/injection unit 450 includes a linear guiding member 454 fixedly connected to the housing member 401, for instance, through the fastening member 453. The linear guiding member can include one or more carriage shafts, a rail system, one or more mechanical extrusions, or a combination thereof. The carriage member 451 is coupled with the linear guiding member and movable along the linear guiding member.

In some embodiments, the syringe plunger movement is provided by actuating the carriage member 451 relative to the body gripping member 452. In some embodiments, the linear actuation of the carriage member is provided by utilizing the actuating member 454 that is driven by the first gear 441, whose operational moments and rotational or translational displacements are compensated by the linear guiding member 455. In some embodiments, this actuation of the carriage member is configured such that the motor driving the system is in-line or collinear with the actuating member, a conveyor belt, one or more pneumatic actuators, one or more hydraulic actuators, or a combination thereof. In some such embodiments, the carriage member is fixed to traverse axially, such as without any or substantially any rotational tendency from the actuating member, on the carriage shaft that are fixed to the housing member 401, the covering member 404, or both.

In some embodiments, when the gripper 400 connects or disconnects the first unit 410 and the second unit 420, the carriage member also moves in the direction parallel to the rotation axis of the first holding member of the first unit (e.g., along the vertical axis) since both functions utilize the same drivetrain. In some such embodiments, controlling a completion speed of each function is fine-tuned through one or more mechanical advantages, such as one or more gear ratios.

In some embodiments, the gripper 400 includes a resistant member 460 to prevent water or other liquid ingress. The resistant member 460 is fixed to the carriage member guided traveling through constraints of the covering member, for example one or more mechanical extrusions, one or more slits 461, one or more rails, one or more zippers, or a combination thereof. In some embodiments, the one or more mechanical extrusions, the one or more slits 461, the one or more rails, the one or more zippers, or the combination thereof includes gasketing material and/or one or more labyrinth seals, one or more shield, or the like.

In some embodiments, one or more sensors, such as one or more linear encoders and/or one or more rotary encoders, are located in the gripper. In some such embodiments, the one or more sensors are configured to detect the linear motion of the carriage member, rotation of the first or second gear, or both to validate the accurate displacement of the carriage member.

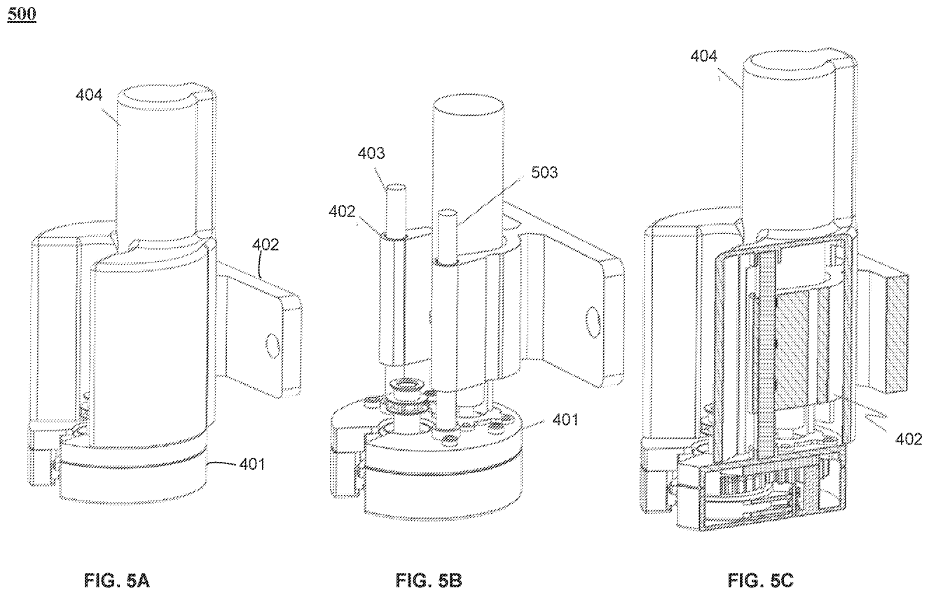

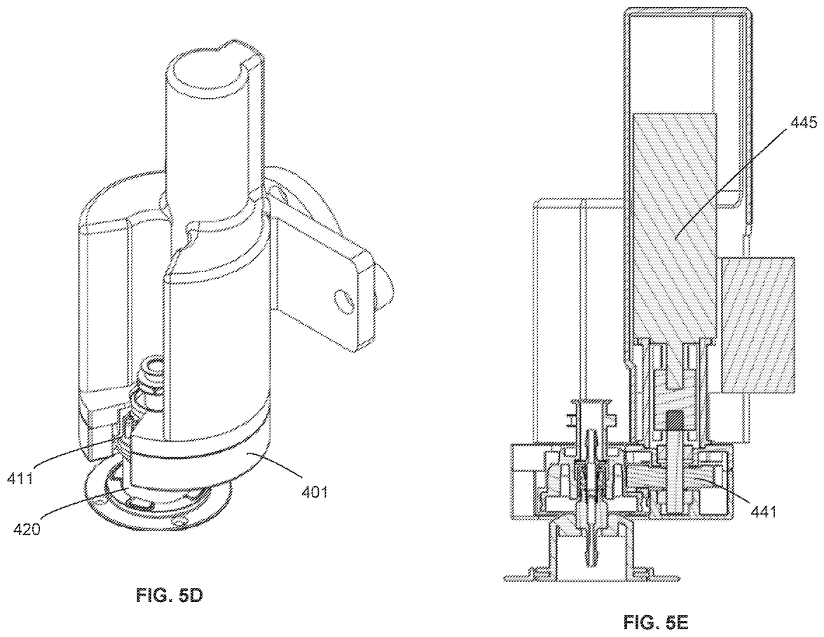

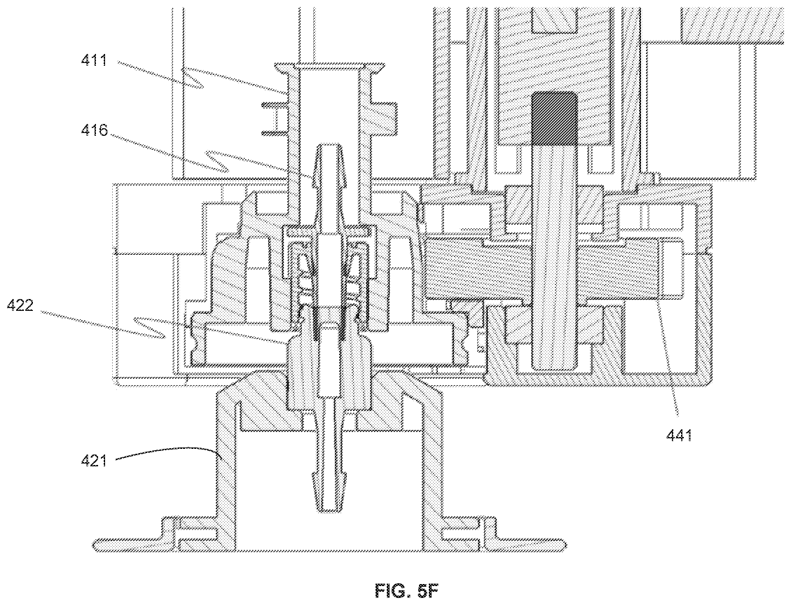

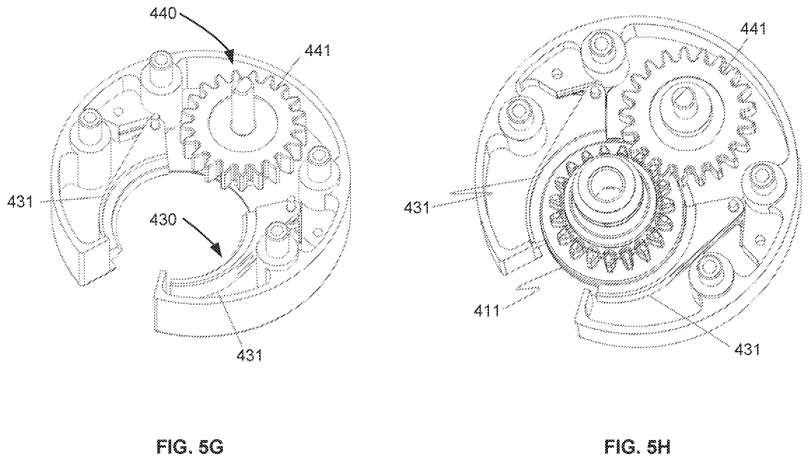

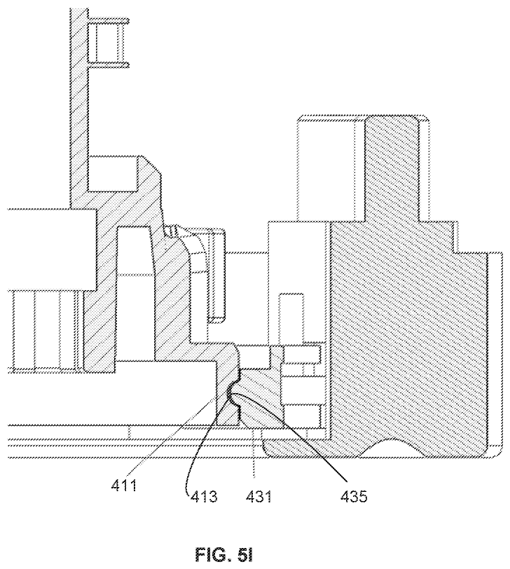

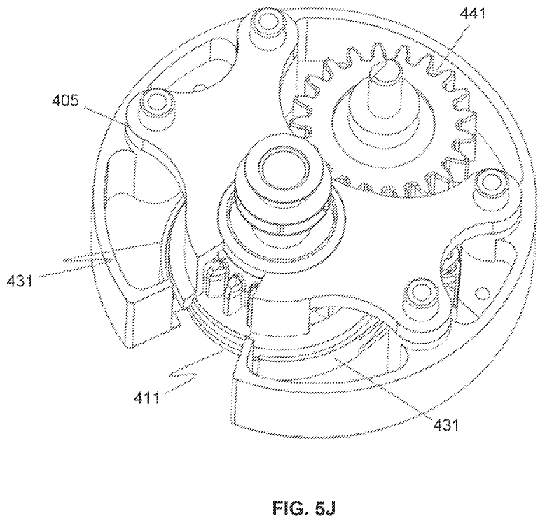

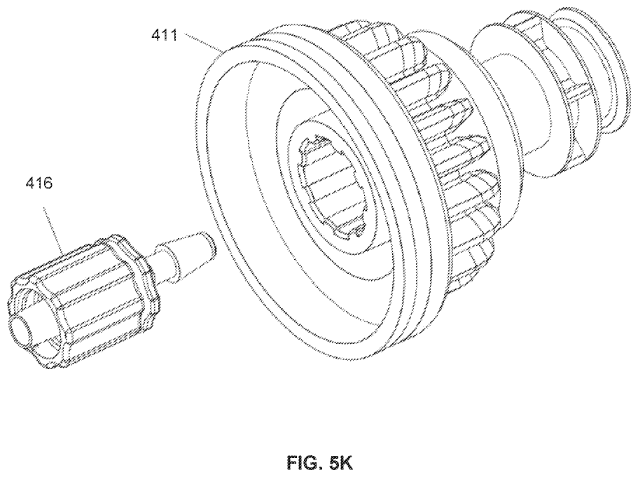

FIGS. 5A-5J illustrate an exemplary gripper 500 in accordance with some embodiments of the present disclosure. The gripper 500 is configured to connect and disconnect connectors, tubes, syringes, or other devices or components that require twisting of a locking element for a successful fluid connection, to grip and transport one or more connectors, tubes, syringes, or other devices or components to different ports of connectors (e.g., self-centering assemblies disclosed herein), or a combination thereof. The gripper 500 can be operated by a robotic arm or other versatile manufacturing equipment.

The gripper 500 is similar to the gripper 400 disclosed herein, except that the gripper 500 does not include a draw/injection unit 450 and the features associated with the draw/injection unit 450. For instance, in some embodiments, the gripper 500 includes a gripping unit 430 and a gear unit 440. In some embodiments, the gripper 500 is configured to connect a first unit 410 with a second unit 420 or disconnecting the first unit from the second unit.

The second unit can be any connector or fluid fitting. For instance, in some embodiments, the second unit is a self-centering assembly, such as the self-centering assembly 100, 200 or 1100 disclosed herein. In a typical application, the second unit is an integral part of an equipment (e.g., a cartridge) or is secured on an equipment or a surface. In some embodiments, the second unit 420 includes a holding member 421 and a fluid fitting member 422.

In some embodiments, the first unit 410 includes a first holding member 411 with an engaging member 413 and a first fitting member 416 fixedly connected to the first holding member. The first fitting member 416 can be a syringe, a tube, a tube fitting member, a connector (e.g., a female or male luer lock connector), a vessel, or any components or devices that can be connected to the second unit (e.g., the fluid fitting member of the second unit).

As a non-limiting example, FIGS. 5E, 5F and 5K illustrate that the first fitting member 416 is a male luer lock connector screwed on the fitting member 422 (a female luer lock connector) of the second unit 420. In some embodiments, the first fitting member 416 (e.g., the male luer lock connector) is housed within and/or fixed to the first holding member 411, for instance, by standard means such as adhesive materials (e.g., one or more strips of adhesive), bonding, thermal welding, or mechanical means (e.g., congruent meshing features). Thus, when the first holding member rotates, the locking feature of the first holding member rotates along with the first holding member. In some embodiments, the first holding member 411 includes one or more locating members 415 (the same or similar to those illustrated in FIGS. 4U and 4V) configured to help locating the first holding member of the first unit with respect to the second unit.

In some embodiments, the gripping unit 430 of the gripper 500 is operable to engage with the first holding member 411 of the first unit 410. In some embodiments, the gripping unit 440 includes a second engaging member 435 matching the first engaging member 413 of the first holding member of the first unit. When the gripping unit is engaged with the first holding member of the first unit, the gripping unit restricts the first holding member of the first unit from moving axially with respect to the gripping unit but allows the first holding member of the first unit to rotate around the first rotation axis of the first holding member. In some embodiments, the gripping unit 430 includes one or more gripping members 431, each rotatable around an axis parallel to the rotation axis of the first holding member of the first unit.

In some embodiments, the gear unit 440 of the gripper 500 includes a first gear 441 operable to rotate the first holding member 411 of the first unit 413 around the first rotation axis. The first gear 441 can be driven by a motor or any other suitable means.

In some embodiments, the gripper 500 includes a capping member, such as a capping member 405 illustrated in FIG. 5J. The capping member is configured to constrain translation movement of the first holding member in a plane normal to the rotation axis of the first holding member, translation movement of the first holding member along the rotation axis of the first holding member, or both. In some embodiments, the capping member serves as a hard stop to define a boundary to prevent the gripping unit from closing further inwardly.

In some embodiments, the gripper 500 includes a housing member 401 configured to accommodate the gripping unit, capping member, first gear, motor, or a combination thereof. In some embodiments, the gripper 500 includes a mounting member 402 for interfacing with an automated equipment (e.g., a robot) and/or for housing the electronics to control the encoders and sensors of the gripper 500. The mounting member 402 and the housing member 401 are movable relative to each other. In some embodiments, the gripper 500 includes one or more shafts 403, each fixedly connected to the housing member 401 and extending along the z-direction. The mounting member 402 is coupled to the one or more shafts 403 and slidable along the one or more shafts 403.

In some embodiments, the gripper 500 includes a covering member 404 to protect some components of the gripper, for instance, to enclose one or more moving parts of the gripper (e.g., the motor), making the gripper easy to clean and resistant to water ingress.

In some embodiments, the axial movement of the first holding member 411 of the first unit 410, the gripping unit 430, the first gear 441 of the gearing unit, the housing member 401, the mounting member 402 with respect to the second unit (e.g., toward or away from the second unit) is detected to ensure proper connection or disconnection.

The operation of the gripper 500 is similar to that of the gripper 400, and description of which is omitted to avoid redundancy.

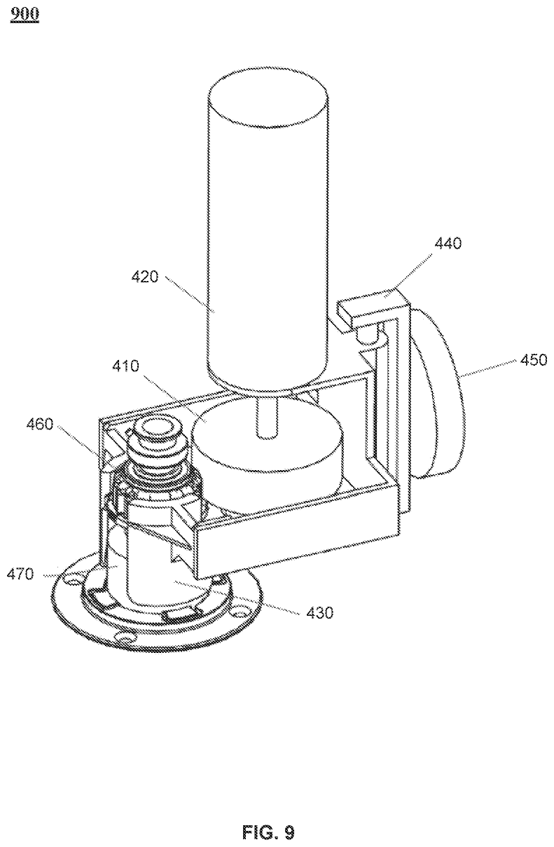

FIG. 9 illustrates an exemplary gripper 900 in accordance with some embodiments of the present disclosure. The gripper 900 is configured to connect and disconnect connectors, tubes, syringes, or other devices or components that require twisting of a locking element for a successful fluid connection, to grip and transport one or more connectors, tubes, syringes, or other devices or components to different ports of connectors (e.g., self-centering assemblies disclosed herein), or a combination thereof. The gripper 900 can be operated by a robotic arm or other versatile manufacturing equipment.

In some embodiments, the gripper 900 includes a gripping unit having one or more gripping members 910, and a gear unit having a first gear, such as the first gear 441 disclosed herein with respect to the gripper 400, 500. In some embodiments, the first gear 411 is driven by the motor 445. In some embodiments, the gripper 900 includes a mounting member 920 for mounting some components of the gripper, guiding movement of some components of the gripper, or both. In some embodiments, the gripper 900 includes a gripper adaptor 930 for interfacing with an automated equipment.

In some embodiments, the gripper 900 is configured to connect a first unit (e.g., the first unit 410 disclosed herein with respect to the gripper 400 or 500) with a second unit (e.g., the second unit 420 disclosed herein with respect to the gripper 400 or 500) or disconnecting the first unit from the second unit. For instance, in some embodiments, the first unit includes a first holding member 411 and a first fitting member, which can be a syringe, a connector (e.g., a male or female luer connector) or any other vessels or connecting components. In some embodiments, the second unit includes a second holding member 421. In some embodiments, the second unit is a self-centering assembly (e.g., the self-centering assembly 100, 200 or 1100 disclosed herein).

In some embodiments, the first fluid fitting member (e.g., a male luer lock connector) is housed within and fixed to the first holding member 411, for instance, by standard means, such as adhesive and mechanical means so that when the first holding member rotates, the first fitting member (e.g., the male luer lock connector) rotates as well. In an alternative embodiment, the thread of the first fitting member (e.g., the male luer lock connector) and features are integral to the first holding member 411 and the component of the first fitting member (e.g., the male luer lock connector) connected to the first holding member can swivel relative to the first holding member. When the first fitting member (e.g., the male luer lock connector) must be unscrewed from the second unit (e.g., the female luer lock connector), the gripper 900 or a portion of the gripper (e.g., the gripping members, gear) is first lowered onto the first holding member so that the center of the one or more gripping members 910 is coincident with the rotating axis of the first holding member.

As the gripper 900 or a portion of the gripper is lowered, before the one or more gripping members interface with the first holding member, the elongated geometry of the one or more gripping members interfaces with the second holding member (e.g., needle free connector housing) 421 which retains and restricts rotational and axial motion of the first fluid fitting member (e.g., the female luer lock connector). Slight misalignments between the one or more gripping members 910 and the second holding member 421 are corrected as the gripper is lowered onto the first holding member 411 because the second holding member is compliant relative to a connection stop. The connection stop, which itself is secured to a relative ground plane, nearly constrains the second holding member, but allows for slight motion rotationally (about the rotational axis of the luer lock connector) and translationally (along the plane normal to the aforementioned axis).

Once the second holding member is aligned with the one or more gripping members 910, the one or more gripping members then begin to interface with the first holding member. The first holding member 411 interferes with the one or more gripping members 910 and flexes due to the shelled and cantilevered geometry of the first holding member.

Once the one or more gripping members 910 are lowered to a certain height relative to the first holding member, the first holding member engages with the one or more gripping members, and is retained axially yet allowed for rotation. The first gear 441 rotates, as driven by the motor 445. In some embodiments, the rotational axis of the motor is colinear with that of the first gear. Both the first holding member and the first gear have involute tooth patterns so that power can be transmitted between them. In an alternative embodiment, power could be transmitted through a different tooth pattern or frictional surface.

As the first gear rotates, it rotates the first holding member. The first holding member and its housed first fitting member (e.g., the male luer lock connector) are unscrewed from the second unit (e.g., the female luer lock connector). Because the first fitting member (e.g., the male luer lock connector) must travel axially away from the second unit (e.g., the female luer lock connector), the first holding member and some or all components of the gripper 900 (e.g., one or more gripping members, first gear, and motor) must move with the first fitting member (e.g., the male luer lock connector). This entire subassembly including one or more gripping members, first gear, and motor float within a mounting member 920 by use of a preloaded spring or some other elastic material and are also guided by a rail or some other component that allows for axial vertical travel yet compensates for off-axis moments. This gives the entire assembly compliance in the axial direction of the first fitting member (e.g., the male luer lock connector). The mounting member is attached to the gripper adapter 930 which allows the assembly to be fastened securely to the end effector of a robot or some other automated equipment.

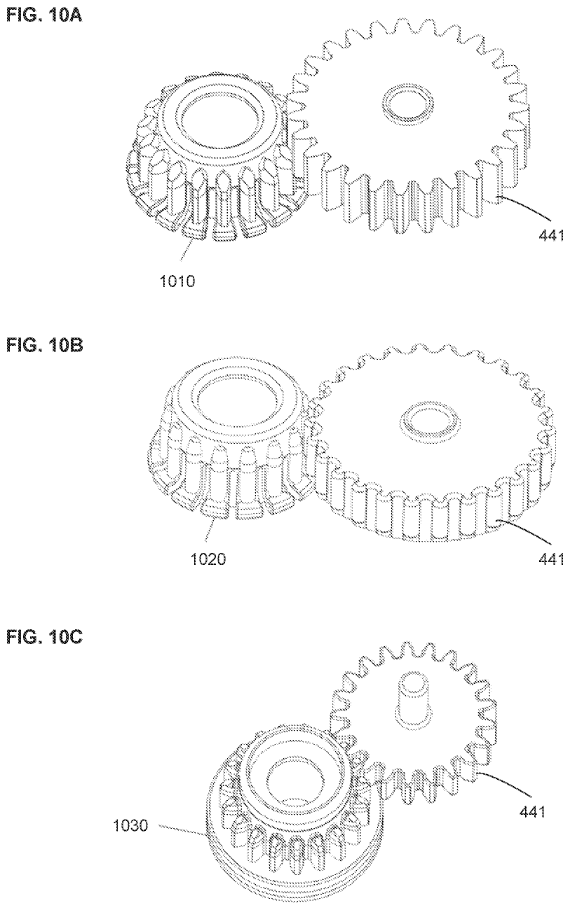

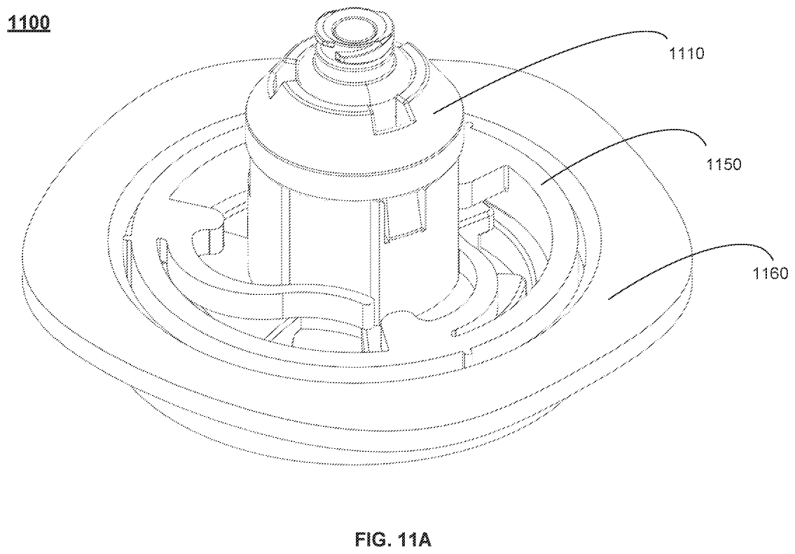

The grippers 400, 500 and 900 disclosed herein or the connecting/disconnecting methods disclosed herein with respect to the grippers 400, 500 and 900 can be extended to make any type of connection (e.g., locking tubing connection) that requires a push-force and/or rotation of one part relative to the other. For instance, as non-limiting examples, FIGS. 10A-10C illustrate connection/disconnection of disinfecting caps 1010, 1020, 1030 to/from the mating connector (e.g., the second unit or the fitting member of the second unit), either male or female.

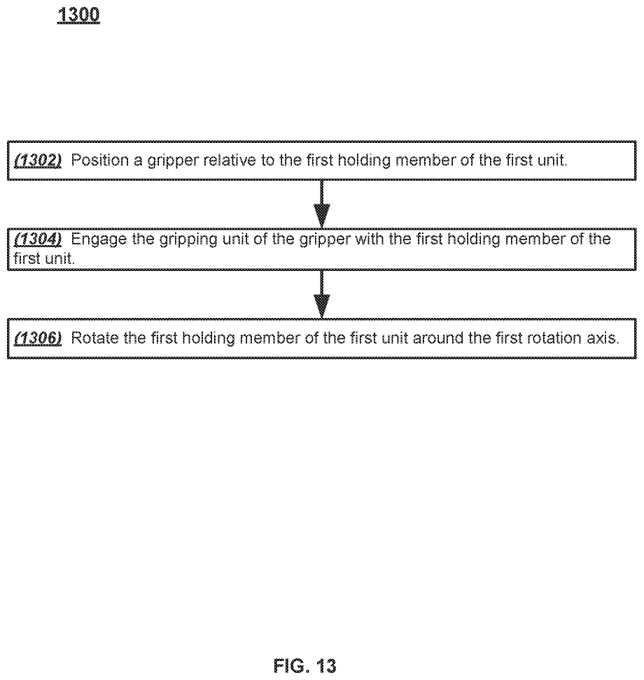

FIG. 13 illustrates an exemplary method 1300 for connecting a first unit with a second unit or disconnecting the first unit from the second unit using a gripper disclosed herein. The first unit includes a first holding member having a first rotation axis. In some embodiments, the first unit includes a syringe or a connector fixedly coupled with the first holding member. In some embodiments, the second unit is a connection unit of a self-centering assembly, such as the self-centering assembly 100, 200 and 1100 disclosed herein.

The method 1300 generally includes positioning a gripper relative to the first holding member of the first unit (block 1302), engaging the gripping unit of the gripper with the first holding member of the first unit (block 1304) and rotating the first holding member of the first unit around the first rotation axis (block 1306).

In some embodiments, the positioning of the gripper relative to the first holding member of the first unit is done by an automated equipment, such as a robot or other automated equipment. In some embodiments, the gripper includes a gripping unit and a gear unit having a first gear, such as those disclosed herein with respect to the gripper 400, 500, 900.

In some embodiments, the engagement of the gripping unit of the gripper with the first holding member of the first unit restricts the first holding member of the first unit from moving axially with respect to the gripping unit of the gripper but allows the first holding member of the first unit to rotate around the first rotation axis.

In some embodiments, the rotating of the first holding member of the first unit around the first rotation axis is via the first gear of the gear unit of the gripper. When the first holding member of the first unit rotates around the first rotation axis, the first holding member of the first unit, the gripping unit and the first gear of the gearing unit move axially with respect to the second unit toward or away from the second unit, thereby connecting the first unit with the second unit or disconnecting the first unit from the second unit.

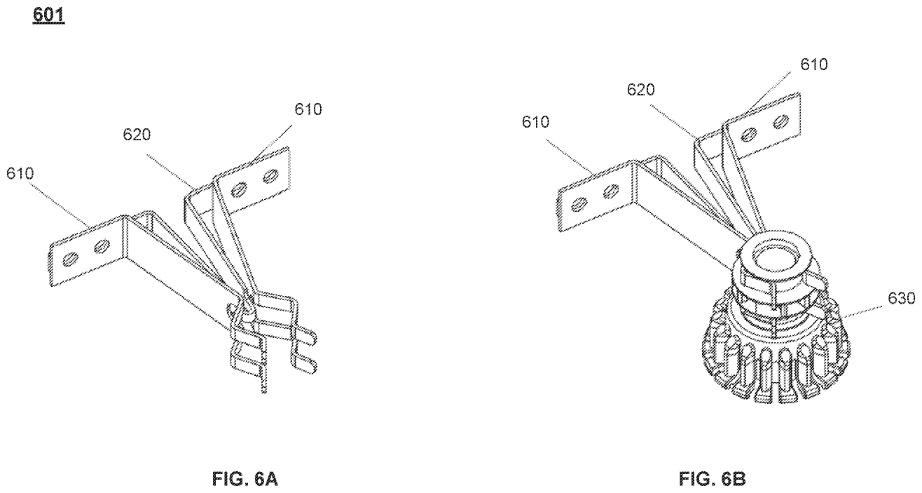

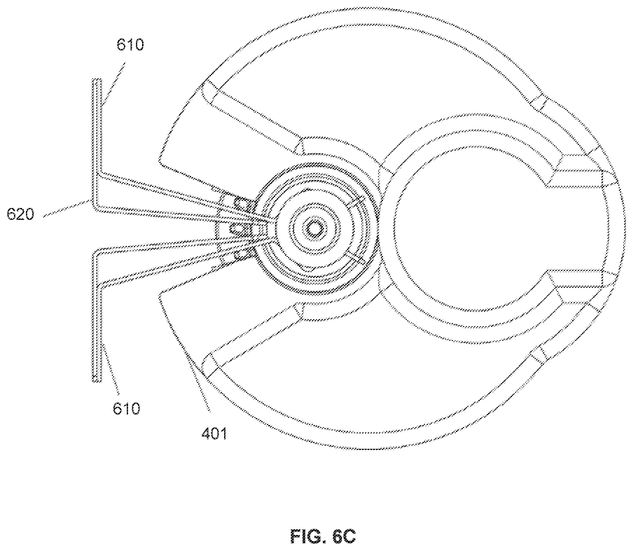

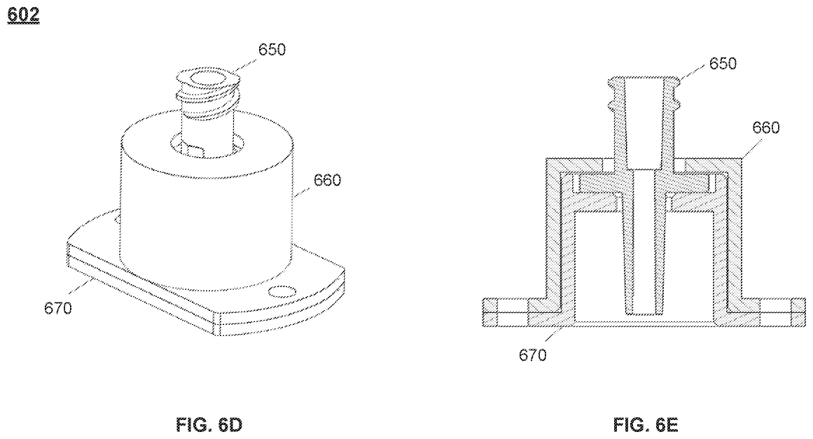

FIGS. 6A-6C illustrate an exemplary clip 601 in accordance with some embodiments of the present disclosure. The clip 601 generally includes one or more clipping members 610, e.g., 1, 2, 3, 4, 5, or more than 5 clipping members. As a non-limiting example, the clip 601 is illustrated with a pair of clipping members. In some embodiments, the clip 601 also includes a stop member 620. In some embodiments, the clip 601 is a wall clip, e.g., it can be mounted on a wall of any equipment.

The clip 601 is configured to retain a holding member 630, which can be any connector or any self-centering assembly disclosed herein, or a component of any connector or any self-centering assembly disclosed herein. For instance, in some embodiments, the function of the one or more clipping members and the stop member is to retain the holding member and maintain its location and orientation between operations. When the holding member needs to be stowed away between operations, the holding member is pressed into the open end of the clipping members and towards the wall on which the clipping members are mounted. The clipping members are compliant against the force used to push the holding member towards the clip, and made to incorporate elastic or compliant structures such as being made out of sheet metal designed to preserve their elastic properties during use. The clipping members expand and then close around the interfacing part of the holding member. Once the clipping members are closed, their shape restricts translation of the holding member along the plane normal to the holding member's rotational axis.

As the holding member is retained translationally, the stop member 620 interfaces with the holding member. In some embodiments, the stop member is a V-shaped part whose vertex is congruent with a series of slots on the holding member and, once coincident with these slots, prevents rotation of the holding member that could otherwise affect future operations. In some embodiments, slots on the holding member are also surrounded by the clipping members above and below, made possible by a slot in the clipping members, therefore constraining the holding member axially.

An alternate embodiment of the clipping members would be some other material whose properties and geometry allow for non-yielding bending and compliance upon insertion of the holding member and adequate retention of the holding member, such as plastic snap cantilevers or spring loaded bearings. The clip stop can be made also to be compliant in the direction toward the wall but still perpendicular to it, so as to constrain the rotation of the holding member but being able to adjust itself and fall into place during the insertion of the holding member by a robot. Also, the features in the holding member mating the clip stop can be designed so as to allow some free rotation of the holding member which can be useful for accommodating random orientations and tolerances of the holding member.

As shown in FIG. 6C, a housing member 401 (e.g., the housing member of the gripper 400, 500) is shaped such that it has clearance around the clipping members when the clipping members are either in, or between, their open and closed positions. This allows for the gripper to take an orthogonal path to the storage and retrieval of the holding member. For instance, to store, the gripper approaches the clipping member directly, on a path that is normal to the surface on which the clipping member is mounted. Once the holding member is secured in the clip, the gripper moves upward on a path parallel to the surface on which the clipping member is secured. For retrieval of the holding member, the path is the reverse of the aforementioned.

FIGS. 6D and 6E illustrate an exemplary clip 602 in accordance with some embodiments of the present disclosure. As a non-limiting example, the clip 602 is illustrated in the form of a female luer lock post. It's function is similar to the clip 601, e.g., to store the holding member between operations while maintaining a relatively similar orientation for ease of retrieval.

In some embodiments, the clip 602 includes a fitting member (e.g., a female luer lock) 650, a retaining member 660 and a housing member 670. When the holding member needs relocation between operations, a gripper screws/tightens the holding member onto the fitting member 650. The housing member 670 constrains the fitting member 650 but allows for rotation compliance about the axis of tightening to compensate for any misalignment between the teeth of the holding member and that of a motor gear when the holding member must be retrieved.

In some embodiments, the housing member 670 also has a hole congruent with the geometry of the fitting member 650 such that it gives the fitting member 650 clearance and translational compliance on the plane normal to the rotation axis of the fitting member 650. The retaining member 660 constrains the fitting member 650 against large axial translations and allows just enough clearance so the fitting member 650 has rotational compliance and translational compliance along the plane normal to the axis of rotation. When the holding member needs to be retrieved, a gripper unscrews the holding member from the female luer Lock Post. This embodiment allows a gripper to approach from any direction for storing and retrieving the holding member.

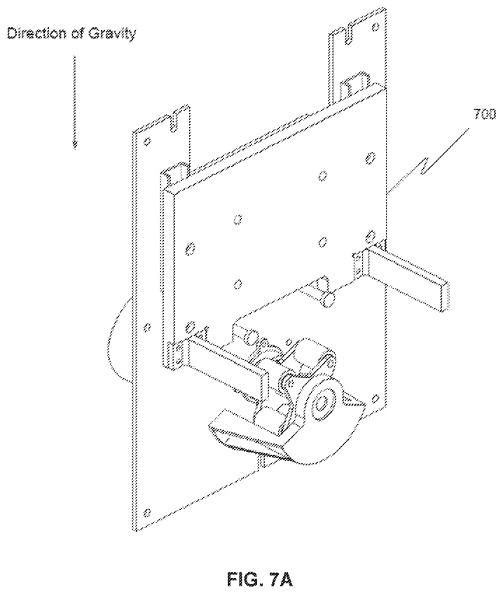

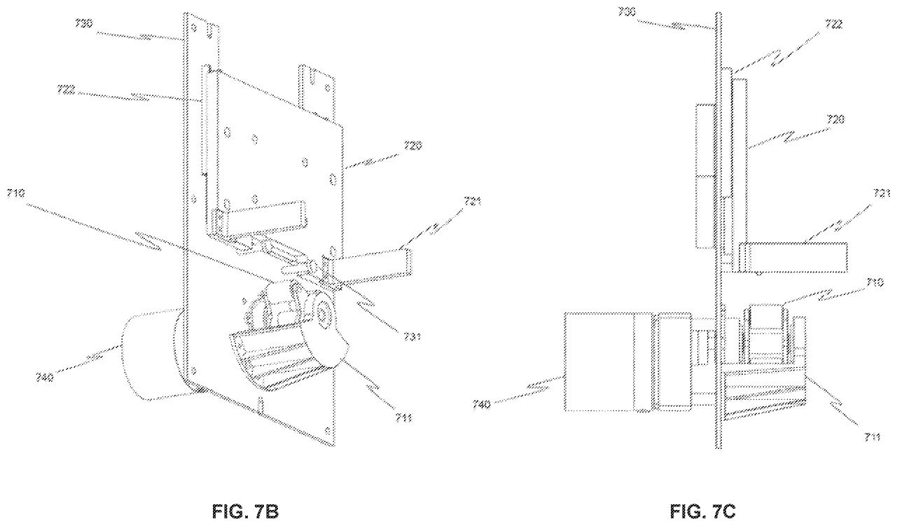

FIGS. 7A-7C illustrate an exemplary peristaltic pump assembly 700 in accordance with some embodiments of the present invention. In some embodiments, the peristaltic pump assembly 700 includes a peristaltic pump head 710 (or “pump head” for short) and a weighted sled 720 (or “sled” for short) that are mounted on a mounting member 730. In some embodiments, the peristaltic pump assembly 700 or the peristaltic pump head 710 includes a bearing housing 711. In some embodiments, the peristaltic pump assembly 700 or the weighted sled 720 includes one or more sled arms 721 and one or more telescopic rails 722. In some embodiments, the peristaltic pump assembly 700 also includes a motor 740.

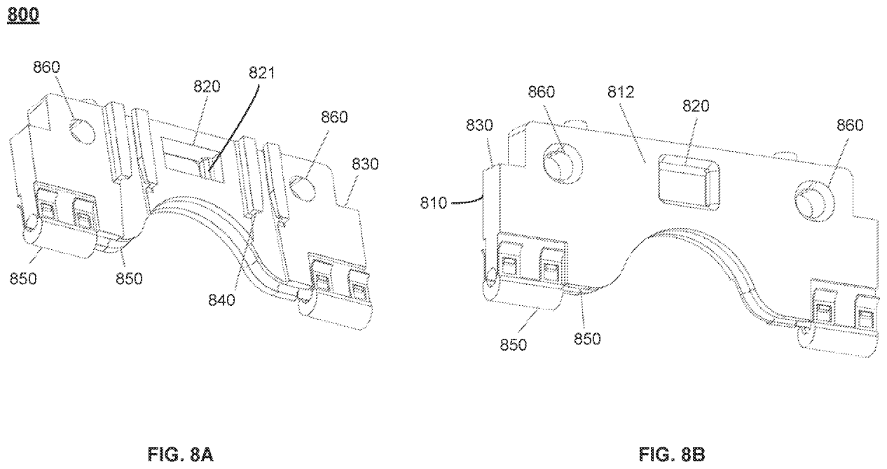

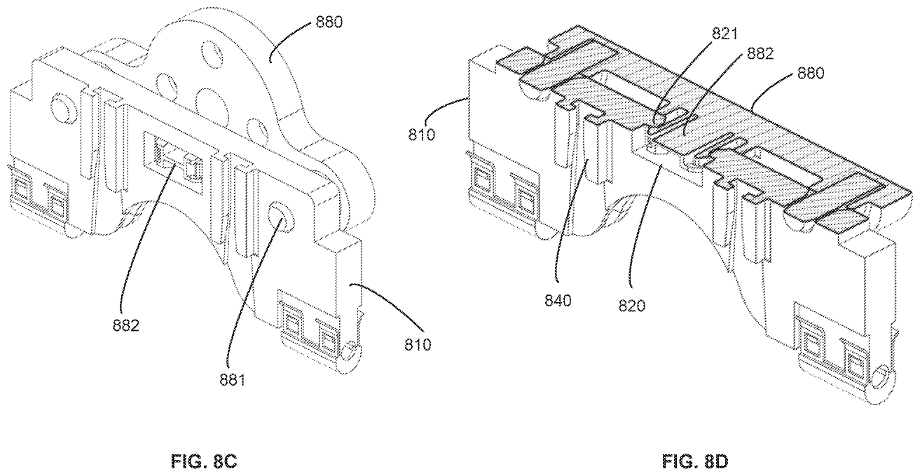

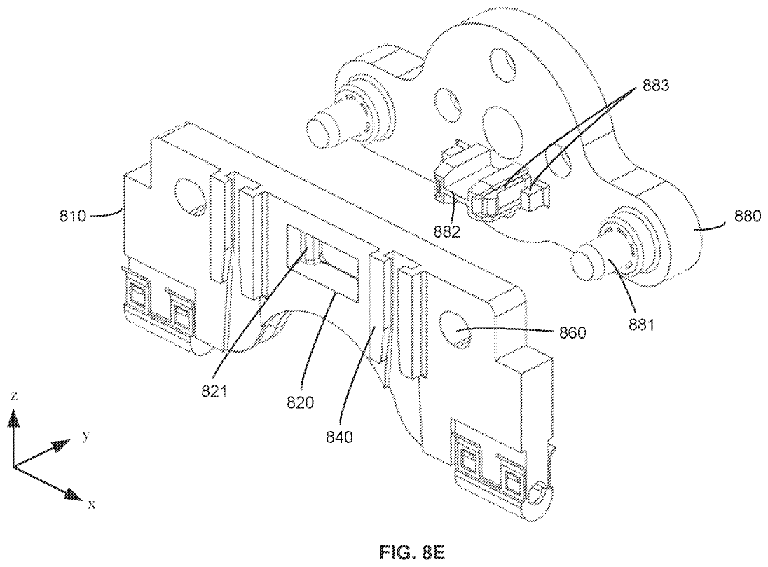

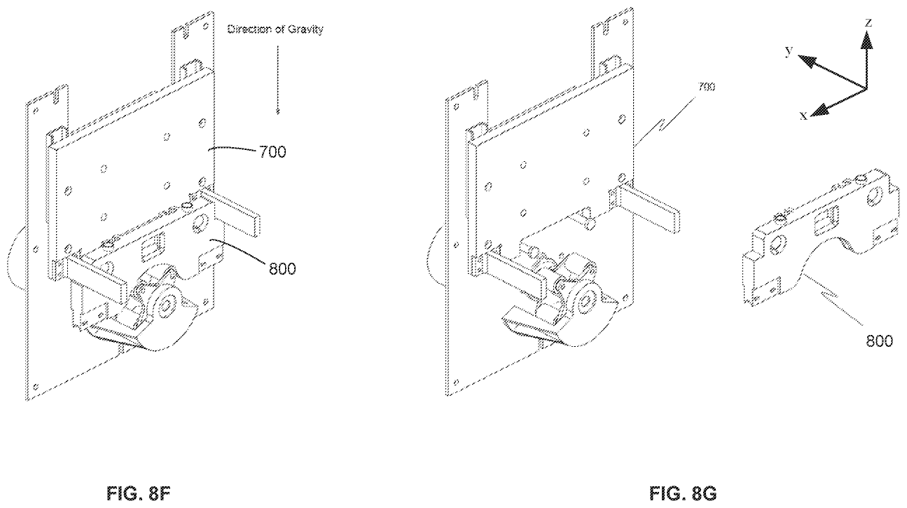

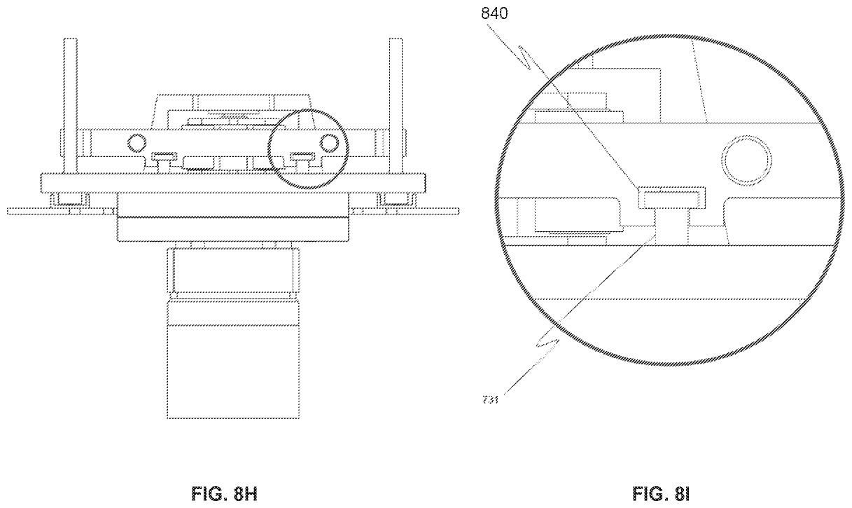

FIGS. 8A-8I illustrates an exemplary cartridge 800 in accordance with some embodiments of the present invention. The cartridge 800 is configured for use with a pump assembly, such as the peristaltic pump assembly 700. In some such embodiments, the cartridge 800 is moved by a robotic arm (e.g., via a gripping member 880) or other versatile manufacturing equipment. In some embodiments, the cartridge 800 includes a cartridge body 810 with a face or surface 812. In some embodiments, the cartridge body 810 is formed with one or more locating members 860 that mates with the one or more matching members 881 of the gripping member 880. In some embodiments, the cartridge body 810 is formed with a locking member 820 for securing the cartridge with the gripping member 880. For instance, in some embodiments, the locking member 820 includes one or more engaging elements 821 that interface with one or more engaging elements of a matching member 882 of the gripping member 880. In some embodiments, the cartridge body 810 is formed with one or more shouldering members 830. In some embodiments, the cartridge 800 includes one or more alignment members 840 formed or coupled with the cartridge body 810. In some exemplary embodiments, the cartridge 800 includes one or more nesting members 850 formed or coupled with the cartridge body 810. As a non-limiting example, two engaging members 860, two shouldering members 830, two alignment members 840, and two nesting members 850 are shown.

In some embodiments, when the cartridge 800 is to be transported, the cartridge is located relative to the gripping member 880 mounted on a robot via the one or more locating members 860 that mate with the one or more matching members 881 of the gripping member 880. In some embodiments, the matching members 881 of the gripping member 880 include locating pins, ramps, wedges, bearing surfaces or similar self-centering or self-aligning features.

The cartridge 800 is secured to the gripping member 880 via the locking member 820 that interface with the matching member 882 of the gripping member 880. In some embodiments, the locking member 820 includes one or more engaging elements 821 that interface with one or more engaging elements 883 of the matching member 882 of the gripping member 880, creating a retention force that retains the cartridge. For instance, in some embodiments, the locking member 820 includes one or more cantilevered snaps or spring loaded protrusions that fit in matching undercuts of the matching member 882 of the gripping member 880 and thus create retention force.

In some embodiments, other features such as locating pins, ramps or bearing surfaces guide and self-center the cartridge while at the same time providing support against operational loads acting in different directions, such as loading the cartridge into the pump assembly. In some such embodiments, the one or more locating members 860 support and constraints all degrees of freedom except the axial motion, as defined perpendicular to the cartridge face 812, which is the direction in which the gripping member 880 approaches and engages with the cartridge. This has the advantage that the locking member 820 only sees loads in the axial direction in which it locks and unlocks to the gripping member 880, which is referred to this cartridge axial direction (e.g., the y-direction illustrated in FIG. 8E).

In some embodiments, when the cartridge 800 is to be loaded into the pump assembly 700, the cartridge 800 first lifts vertically (e.g., along the z-direction illustrated in FIG. 8G) in the weighted sled 720 upward and the cartridge 800 is moved inward horizontally (e.g., along the y-direction illustrated in FIG. 8G) so that it is situated directly above the peristaltic pump head 710. In some embodiments, the inward movement of the cartridge 800 is either simultaneous or sequential with the upward movement that lifts the sled, so long as the cartridge 800 does not collide with the pump head in this motion.

In some embodiments, the cartridge 800 is moved inward and high enough relative to the mounting member 730 that the one or more alignment members 840 of the cartridge 800 is lowered onto and engaged with the one or more pump mating members 731. Once engaged, these features ensure that the cartridge is properly located on the pump head and secure the cartridge in the horizontal direction. They also compensate for any non-vertical directional forces created by the pump head operation. In some embodiments, this is achieved through the use of T-slots and shoulder screws, which also act as a hard stop for the sled when the cartridge is not loaded.

The advantage of the orthogonality between the vertical motion of the cartridge loading and the horizontal motion of the robot gripping the cartridge allows the robot to grip it with passive means, such as snaps, as opposed to stronger more complicated means such as actuated grippers.

When lifting the sled, the one or more shouldering members 830 of the cartridge 800 interface with the one or more sled arms 721. The one or more sled arms and the one or more shouldering members are geometrically shaped and spaced in such a way that, when interfacing, the cartridge is in line with the pump head given manufacturing and operational tolerances. In some embodiments, this is achieved through locating pins, ramps, or other bearing surfaces.

In the lifting of the sled, the one or more telescopic rails 722 both constrain the sled's movements to the vertical direction and compensate for any off-axis moments from the sled's weight and/or its interactions with cartridge and/or pump. In some embodiments, the constraint includes lead screws, 4-bar mechanisms, articulated hinges or other linear guides that allow the sled to preload the cartridge by gravity.

In some embodiments, when the cartridge moves inward toward the sled, it is done under the load of the sled, and thus the friction and potential wear between the sled arms and cartridge shoulders is reduced by controlling said interface, e.g., attaching low-friction pads or using rolling features like bearings.

The one or more nesting members 850 are configured to nest and secure a flexible tube used in peristalsis. In some embodiments, the one or more nesting members 850 include grooves, holes, snaps, magnet fitted caps, hinged caps, etc. The cartridge 800 also has a geometry, which the tube runs along, that is congruent with the rotating path of the pump head. Once the cartridge is resting atop the pump head under the weight of the sled, and thus compressing the flexible tube between the cartridge and the pump head, the motor 740, whose rotating axis is collinear with that of the pump head, drives the pump head which rotates it and creates peristalsis in the cartridge's tube. Because the pump head experiences radial forces from the peristalsis's compressive force, these forces are compensated by the pump head's bearing system, which includes a bearing housing 711 that has enough clearance to allow for the cartridge to rest on the pump head.

In some embodiments, the pump assembly relies on gravity to provide the compressive force between the rotating pump head and the cartridge's tube. The advantage of this configuration is that the compressive force is consistent and controlled throughout operation and does not require other power driven mechanisms or systems. In some embodiments, the compressive force is provided by a more space efficient passive mechanism, like springs or elastic elements, or power driven mechanisms, like pneumatics or hydraulics actuators, motor driven actuators or other types of actuators, with or without force sensing capabilities.

In some embodiments, once peristalsis and pumping is complete, the cartridge is unloaded via movements that are the reverse of its loading.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces disclosed herein. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A self-centering assembly comprising:
a connection unit comprising a holding member, wherein the holding member comprises a side wall, and the side wall comprises a plurality of contoured surface areas arranged circumferentially; and
a centering unit configured to couple the connection unit with a base unit, wherein the centering unit comprises a plurality of contoured elastic members arranged circumferentially along an axis of the centering unit, and each respective contoured elastic member in the plurality of elastic members matches a corresponding contoured surface area in the plurality of contoured surface areas on the side wall of the connection unit, thereby allowing the connection unit to be displaced with respect to the axis of the centering unit when the connection unit is subject to an external force and forcing the connection unit to return to its original position when the external force is removed.

2. The self-centering assembly of claim 1, wherein the base unit is an integral part of a device, or is fixedly coupled with the device.

3. The self-centering assembly of claim 2, wherein the device is a cartridge.

4. The self-centering assembly of any preceding claim, further comprising the base unit.

5. The self-centering assembly of any preceding claim, wherein
the base unit comprises a shouldering member;
the holding member of the connection unit sits on the shouldering member of the base unit;
one or more slots are formed at one of the shouldering member of the base unit and the holding member of the connection unit; and
one or more protrusions are formed at the other of the shouldering member of the base unit and the holding member of the connection unit,
wherein the one or more slots and the one or more protrusions are coupled with each other to define a maximal allowed displacement of the connection unit with respect to the base unit.

6. The self-centering assembly of claim 5, wherein:
the holding member of the connection unit comprises a flange sitting on the shouldering member of the base unit.

7. The self-centering assembly of claim 6, wherein:
the one or more slots are formed at the shouldering member of the base unit;
the one or more protrusions are protruded from the flange of the holding member of the connection unit.

8. The self-centering assembly of any one of claims 6-7, wherein:
the centering unit comprises a plurality of overhangs arranged circumferentially, each sitting on the flange of the holding member of the connection unit to prevent axial movement of the connection unit with respect to the centering unit or the base unit.

9. The self-centering assembly of claim 8, wherein the number of the overhangs is the same as the number of contoured elastic members.

10. The self-centering assembly of claim 8, wherein the number of the overhangs is different than the number of contoured elastic members.

11. The self-centering assembly of any one of claims 8-10, wherein the side wall of the holding member of the connection unit further comprises a plurality of slots, each corresponding to an overhang in the plurality of overhangs to provide space for the overhang when the connection unit is displaced.

12. The self-centering assembly of any preceding claim, wherein the centering unit is fixedly coupled with the base unit.

13. The self-centering assembly of claim 12, wherein the centering unit is fixedly coupled with the base unit by snap-fitting.

14. The self-centering assembly of any preceding claim, wherein the side wall is cylindrical.

15. The self-centering assembly of any preceding claim, wherein the connection unit further comprises a fluid fitting member fixedly coupled with the holding member.

16. The self-centering assembly of claim 15, wherein the fluid fitting member is a luer-lock connector.

17. The self-centering assembly of any one of claims 15-16, wherein the holding member of the connection unit comprises:
a first holding piece sitting on the base unit; and
a second holding piece fixedly coupled with the first holding piece and configured to secure the fluid fitting member in place with respect to the first holding piece.

18. The self-centering assembly of claim 17, wherein the first and second holding pieces are coupled with each other by snap-fitting.

19. The self-centering assembly of any preceding claim, wherein adjacent contoured surface areas in the plurality of contoured surface areas are spaced apart from each other.

20. The self-centering assembly of any preceding claim, wherein contoured surface areas in the plurality of contoured surface areas are substantially uniformly distributed in a circumferential direction.

21. The self-centering assembly of any preceding claim, wherein the plurality of contoured surface areas comprises at least 2, at least 3, at least 4, or at least 5 contoured surface areas.

22. The self-centering assembly of any preceding claim, wherein each contoured surface area is a concave area on the side wall of the holding member of the connection unit.

23. A self-centering assembly comprising:
a connection unit comprising a holding member, wherein the holding member comprises a cylindrical side wall; and
a centering unit configured to couple the connection unit to a device, wherein (i) the centering unit comprises a plurality of elastic members arranged circumferentially along an axis of the centering unit, and (ii) each of the plurality of elastic members forms a contact with the cylindrical side wall of the holding member of the connection unit, thereby allowing the connection unit to be displaced with respect to the axis of the centering unit when the connection unit is subject to an external force and forcing the connection unit to return to its original position when the external force is removed.

24. The self-centering assembly of claim 23, wherein:
the holding member of the connection unit comprises a flange; and
the centering unit comprises a plurality of overhangs arranged circumferentially, each sitting on the flange of the holding member of the connection unit to prevent axial movement of the connection unit with respect to the centering unit.

25. The self-centering assembly of any one of claims 23-24, wherein:
one or more slots are formed at one of the connection unit and the centering unit; and
one or more protrusions are formed at the other of the connection unit and the centering unit,
wherein the one or more slots and the one or more protrusions are coupled with each other to define a maximal allowed displacement of the connection unit with respect to the centering unit.

26. The self-centering assembly of any one of claims 23-25, wherein the connection unit further comprises a fluid fitting member fixedly coupled with the holding member.

27. The self-centering assembly of claim 26, wherein the fluid fitting member is a luer-lock connector.

28. A cartridge comprising:
a cartridge body comprising one or more first locking members and one or more second locking members, wherein the one or more first locking members and one or more second locking members are substantially orthogonal to each other and configured to lock the cartridge with a gripper or an equipment; and
one or more connectors disposed on the cartridge body and connected to one or more containers secured by the cartridge, wherein each of the one or more connectors is configured for transferring a fluid to or from a container in the one or more containers.

29. The cartridge of claim 28, wherein the container in the one or more containers is made of a flexible material.

30. The cartridge of claim 29, wherein the container in the one or more containers is a bag.

31. The cartridge of claim 28, wherein the container in the one or more containers is made of a rigid material.

32. The cartridge of claim 31, wherein the container in the one or more containers is a flask.

33. The cartridge of any one of claims 28-32, wherein:
the cartridge body comprises a first side and a second side opposite to the first side;
a corresponding first locking member in the one or more first locking members and a corresponding second locking member in the one or more second locking members are disposed on each of the first and second sides of the cartridge body; and
the corresponding first locking member is substantially orthogonal to the corresponding second locking member.

34. The cartridge of claim 33, wherein for each of the first and second sides of the cartridge body:
one of the corresponding first and second locking members comprises a cantilevered snap or a spring loaded protrusion; and
the other of corresponding first and second locking members comprises a slot.

35. The cartridge of any one of claims 28-34, further comprising:
a retaining member for retaining the container;
a reference tag;
a bar code tag;
one or more electronics connectors;
one or more additional connectors;
one or more sensors;
one or more sensor pads; or
any combination thereof.

36. The cartridge of any one of claims 28-35, wherein a respective connector in the one or more connectors is a self-centering assembly, and the self-centering assembly comprises:
a corresponding connection unit; and
a corresponding centering unit coupled with the corresponding connection unit and configured to allow the corresponding connection unit to be displaced with respect to an axis of the corresponding centering unit when the corresponding connection unit is subject to an external force and force the corresponding connection unit to return to its original position when the external force is removed.

37. The cartridge of claim 36, wherein the corresponding centering unit couples the corresponding connection unit with a corresponding base unit that is integrally formed or fixedly coupled with a body of the cartridge.

38. The cartridge of any one of claims 36-37, wherein:
the corresponding connection unit comprises a holding member, wherein the holding member comprises a side wall, and the side wall comprises a plurality of contoured surface areas arranged circumferentially; and
the corresponding centering unit comprises a plurality of contoured elastic members arranged circumferentially along an axis of the centering unit, and each respective contoured elastic member in the plurality of elastic members matches a corresponding contoured surface area in the plurality of contoured surface areas on the side wall of the connection unit, thereby allowing the connection unit to be displaced with respect to the axis of the centering unit when the connection unit is subject to an external force and forcing the connection unit to return to its original position when the external force is removed.

39. The cartridge of claim 38, wherein:
the corresponding base unit comprises a shouldering member;
the holding member of the corresponding connection unit sits on the shouldering member of the corresponding base unit;
one or more slots are formed at one of the shouldering member of the corresponding base unit and the holding member of the corresponding connection unit; and
one or more protrusions are formed at the other of the shouldering member of the corresponding base unit and the holding member of the corresponding connection unit,
wherein the one or more slots and the one or more protrusions are coupled with each other to define a maximal allowed displacement of the corresponding connection unit with respect to the corresponding base unit.

40. The cartridge of claim 39, wherein:
the holding member of the corresponding connection unit comprises a flange sitting on the shouldering member of the corresponding base unit.

41. The cartridge of claim 40, wherein:
the one or more slots are formed at the shouldering member of the corresponding base unit;
the one or more protrusions are protruded from the flange of the holding member of the corresponding connection unit.

42. The cartridge of any one of claims 40-41, wherein:
the corresponding centering unit comprises a plurality of overhangs arranged circumferentially, each sitting on the flange of the holding member of the corresponding connection unit to prevent axial movement of the corresponding connection unit with respect to the corresponding centering unit or the corresponding base unit.

43. The cartridge of claim 42, wherein the side wall of the holding member of the corresponding connection unit further comprises a plurality of slots, each corresponding to an overhang in the plurality of overhangs to provide space for the overhang when the corresponding connection unit is displaced with respect to the axis of the corresponding centering unit.

44. The cartridge of any one of claims 36-37, wherein:
the corresponding connection unit comprising a holding member, wherein the holding member comprises a cylindrical side wall; and
the corresponding centering unit comprises a plurality of elastic members arranged circumferentially along an axis of the centering unit, wherein each of the plurality of elastic members forms a contact with the cylindrical side wall of the holding member of the corresponding connection unit, thereby allowing the corresponding connection unit to be displaced with respect to the axis of the corresponding centering unit when the corresponding connection unit is subject to an external force and forcing the corresponding connection unit to return to its original position when the external force is removed.

45. The cartridge of claim 44, wherein:
the holding member of the corresponding connection unit comprises a flange; and
the corresponding centering unit comprises a plurality of overhangs arranged circumferentially, each sitting on the flange of the holding member of the corresponding connection unit to prevent axial movement of the connection unit with respect to the corresponding centering unit.

46. The cartridge of any one of claims 44-45, wherein:
one or more slots are formed at one of the corresponding connection unit and the corresponding centering unit; and
one or more protrusions are formed at the other of the corresponding connection unit and the corresponding centering unit,
wherein the one or more slots and the one or more protrusions are coupled with each other to define a maximal allowed displacement of the corresponding connection unit with respect to the corresponding centering unit.

47. The cartridge of any one of claims 36-46, wherein the corresponding connection unit further comprises a fluid fitting member fixedly coupled with the holding member.

48. The cartridge of claim 47, wherein the fluid fitting member is a luer-lock connector.

49. A cartridge comprising:
a cartridge body;
a first locking member formed or coupled with the cartridge body, and configured to interface with a second locking member of a gripping member of a gripper to lock the cartridge body relative to the gripping member of the gripper in a first direction; and
one or more locating members formed or coupled with the cartridge body, and configured to interface with one or more matching members of the gripping member of the gripper to restrict motion of the cartridge body relative to the gripping member of the gripper in a plane substantially orthogonal to the first direction.

50. The cartridge of claim 49, further comprising:
one or more alignment members configured to interface with one or more mating members of a pump assembly, wherein the one or more alignment members allow the cartridge to be loaded to the pump assembly in a direction substantially perpendicular to the first direction.

51. The cartridge of any one of claims 49-50, further comprising:
one or more nesting members configured to nest and secure a flexible tube used in peristalsis.

52. A gripper for connecting a first unit with a second unit or disconnecting the first unit from the second unit, wherein the first unit comprises a first holding member having a first rotation axis, the gripper comprising:
a gripping unit operable to engage with the first holding member of the first unit, wherein when the gripping unit is engaged with the first holding member of the first unit, the gripping unit restricts the first holding member of the first unit from moving axially with respect to the gripping unit but allows the first holding member of the first unit to rotate around the first rotation axis; and
a gear unit comprising a first gear operable to rotate the first holding member of the first unit around the first rotation axis, wherein when the first holding member of the first unit rotates around the first rotation axis, the first holding member of the first unit, the gripping unit and the first gear of the gearing unit move axially with respect to the second unit toward or away from the second unit, thereby connecting the first unit with the second unit or disconnecting the first unit from the second unit.

53. The gripper of claim 52, wherein:
the first holding member of the first unit comprises a first engaging member at an exterior side thereof; and
the gripping unit comprises a second engaging member at an interior side thereof that matches the first engaging member of the first holding member of the first unit.

54. The gripper of claim 53, wherein:
one of the first and second engaging members is a groove; and
the other of the first and second engaging members is a protrusion that fits with the groove.

55. The gripper of any one of claims 52-54, wherein the gripping unit comprises one or more gripping members, each rotatable around an axis parallel to the rotation axis of the first holding member of the first unit.

56. The gripper of claim 55, wherein the one or more gripping members comprises a first gripping member and a second gripping member symmetrical with respect to each other.

57. The gripper of any one of claims 52-56, wherein when the gripping unit is engaged with the first holding member of the first unit, the gripping member defines a gripping axis that coincides with the rotation axis of the first holding member of the first unit.

58. The gripper of any one of claims 52-57, wherein:
the axial movement of the first holding member of the first unit, the gripping unit or the first gear of the gearing unit with respect to the second unit is detected to ensure correct engagement or disengagement of the first and second units.

59. The gripper of claim 58, wherein the detected movement is correlated to angular rotation of a motor to validate the correct engagement or disengagement of the first and second units.

60. The gripper of any one of claims 52-59, wherein the first holding member of the first unit comprises a first tooth pattern and the first gear of the gear unit comprises a second tooth pattern matching the first tooth pattern.

61. The gripper of any one of claims 52-60, wherein the first holding member of the first unit comprises one or more locating members at a side facing the second unit when the first unit is brought toward the second unit and configured to help locating the first holding member of the first unit with respect to the second unit.

62. The gripper of claim 61, wherein the one or more locating members comprise a plurality of extrusions disposed circumferentially, thereby assisting in centering the first holding member with respect to the second unit.

63. The gripper of any one of claims 52-62, further comprising a capping member configured to constrain translation movement of the first holding member in a plane normal to the rotation axis of the first holding member, translation movement of the first holding member along the rotation axis of the first holding member, or both.

64. The gripper of claim 63, wherein the capping member serves as a hard stop to define a boundary to prevent the gripping unit from closing further inwardly.

65. The gripper of any one of claims 52-64, wherein:
the first unit further comprises a first fitting member fixedly connected to the first holding member;
the second unit comprises a second fitting member;
connecting the first unit with the second unit locks the first fitting member with the second fitting member of the second unit, thereby forming a path for transferring a fluid; and
disconnecting the first unit from the second unit unlocks the first fitting member from the second fitting member.

66. The gripper of claim 65, wherein the first fitting member of the first unit is a syringe, a tubing, a connector, or a vessel.

67. The gripper of any one of claims 65-66, wherein
one of the first fitting member of the first unit and the second fitting member of the second unit includes a male luer lock; and
the other of the first fitting member of the first unit and the second fitting member of the second unit includes a female luer lock.

68. The gripper of any one of claims 52-67, wherein the second unit is a corresponding connection unit of a self-centering assembly.

69. The gripper of claim 68, wherein the self-centering assembly comprises:
a corresponding centering unit coupled with the corresponding connection unit and configured to allow the corresponding connection unit to be displaced with respect to an axis of the corresponding centering unit when the corresponding connection unit is subject to an external force and force the corresponding connection unit to return to its original position when the external force is removed.

70. The gripper of claim 69, wherein the corresponding centering unit couples the corresponding connection unit with a corresponding base unit that is integrally formed or fixedly coupled with a body of the gripper.

71. The gripper of any one of claims 69-70, wherein:
the holding member of the corresponding connection unit comprises a side wall, and the side wall comprises a plurality of contoured surface areas arranged circumferentially; and
the corresponding centering unit comprises a plurality of contoured elastic members arranged circumferentially along an axis of the centering unit, and each respective contoured elastic member in the plurality of elastic members matches a corresponding contoured surface area in the plurality of contoured surface areas on the side wall of the connection unit, thereby allowing the connection unit to be displaced with respect to the axis of the centering unit when the connection unit is subject to an external force and forcing the connection unit to return to its original position when the external force is removed.

72. The gripper of claim 71, wherein:
the corresponding base unit comprises a shouldering member;
the holding member of the corresponding connection unit sits on the shouldering member of the corresponding base unit;
one or more slots are formed at one of the shouldering member of the corresponding base unit and the holding member of the corresponding connection unit; and
one or more protrusions are formed at the other of the shouldering member of the corresponding base unit and the holding member of the corresponding connection unit,
wherein the one or more slots and the one or more protrusions are coupled with each other to define a maximal allowed displacement of the corresponding connection unit with respect to the corresponding base unit.

73. The gripper of claim 72, wherein:
the holding member of the corresponding connection unit comprises a flange sitting on the shouldering member of the corresponding base unit.

74. The gripper of claim 73, wherein:
the one or more slots are formed at the shouldering member of the corresponding base unit;
the one or more protrusions are protruded from the flange of the holding member of the corresponding connection unit.

75. The gripper of any one of claims 72-73, wherein:
the corresponding centering unit comprises a plurality of overhangs arranged circumferentially, each sitting on the flange of the holding member of the corresponding connection unit to prevent axial movement of the corresponding connection unit with respect to the corresponding centering unit or the corresponding base unit.

76. The gripper of claim 75, wherein the side wall of the holding member of the corresponding connection unit further comprises a plurality of slots, each corresponding to an overhang in the plurality of overhangs to provide space for the overhang when the corresponding connection unit is displaced with respect to the axis of the corresponding centering unit.

77. The gripper of any one of claims 69-70, wherein:
the holding member of the corresponding connection unit comprises a cylindrical side wall; and
the corresponding centering unit comprises a plurality of elastic members arranged circumferentially along an axis of the centering unit, wherein each of the plurality of elastic members forms a contact with the cylindrical side wall of the holding member of the corresponding connection unit, thereby allowing the corresponding connection unit to be displaced with respect to the axis of the corresponding centering unit when the corresponding connection unit is subject to an external force and forcing the corresponding connection unit to return to its original position when the external force is removed.

78. The gripper of claim 77, wherein:
the holding member of the corresponding connection unit comprises a flange; and
the corresponding centering unit comprises a plurality of overhangs arranged circumferentially, each sitting on the flange of the holding member of the corresponding connection unit to prevent axial movement of the connection unit with respect to the corresponding centering unit.

79. The gripper of any one of claims 77-78, wherein:
one or more slots are formed at one of the corresponding connection unit and the corresponding centering unit; and
one or more protrusions are formed at the other of the corresponding connection unit and the corresponding centering unit,
wherein the one or more slots and the one or more protrusions are coupled with each other to define a maximal allowed displacement of the corresponding connection unit with respect to the corresponding centering unit.

80. The gripper of any one of claims 52-79, further comprising:
a housing member configured to accommodate the gripping unit, the first gear, a second gear of the gear unit, a capping member, a motor, or a combination thereof.

81. The gripper of claim 80, further comprising:
a mounting member for interfacing with an automated equipment, wherein the mounting member and the housing member are axially movable relative to each other.

82. The gripper of claim 81, further comprising:
one or more shafts, each fixedly connected to the housing member and extending axially, wherein the mounting member is coupled to the one or more shafts and slidable along the one or more shafts.

83. The gripper of any one of claims 80-82, further comprising:
a covering member to enclose one or more moving parts of the gripper.

84. The gripper of any one of claims 52-83, further comprising:
a draw/injection unit configured to draw or inject a fluid into or from a syringe, wherein the draw/injection unit comprises:
a body gripping member configured to interface with a body of the syringe; and
a carriage member configured to interface with a plunger of the syringe, and movable relative to the body gripping member in a plunger displacement direction.

85. The gripper of claim 84, wherein the syringe is the first fitting member of the first unit.

86. The gripper of any one of claims 84-85, wherein the draw/injection unit comprises:
an actuating member to actuate a movement of the carriage member.

87. The gripper of claim 86, wherein the actuating member is a screw driven by the first gear of the gear unit.

88. The gripper of any one of claims 84-87, wherein the draw/injection unit comprises:
a linear guiding member to guide a movement of the carriage member, wherein the carriage member is coupled with the linear guiding member and movable along the linear guiding member.

89. The gripper of any one of claims 84-88, wherein the draw/injection unit comprises:
one or more hard stops, each configured to define a lowest or highest point that the carriage member can travel.

90. A gripper for connecting a first unit with a second unit or disconnecting the first unit from the second unit, wherein the first unit comprises a first holding member having a first rotation axis and the second unit comprises a second holding member, the gripper comprising:
one or more gripping members operable to engage with the first holding member of the first unit and the second holding member of the second unit, wherein when the one or more gripping members are engaged with the first holding member of the first unit and the second holding member of the second unit, the one or more gripping members (i) restrict axial motion of the first holding member of the first unit relative to the one or more gripping members, (ii) allow rotational motion of the first holding member of the first unit relative to the one or more gripping members around the first rotation axis; and (iii) restrict rotational motion of the second holding member of the second unit relative to the one or more gripping members; and
a first gear operable to rotate the first holding member of the first unit around the first rotation axis to connect the first unit with the second unit or disconnect the first unit from the second unit.

91. The gripper of claim 90, wherein the second unit is a connection unit of a self-centering assembly.

92. The gripper of any one of claims 90-91, wherein the first unit comprises a first fluid fitting member fixedly coupled with the first holding member.

93. The gripper of claim 92, wherein the first fluid fitting member is a male or female luer connector.

94. The gripper of any one of claims 90-93, wherein the one or more gripping members collectively define a gripping axis that coincides with the rotation axis of the first holding member of the first unit.

95. A method for connecting a first unit with a second unit or disconnecting the first unit from the second unit, wherein the first unit comprises a first holding member having a first rotation axis, the method comprising:
positioning, using an automated equipment, a gripper relative to the first holding member of the first unit, wherein the gripper comprises a gripping unit and a gear unit, and the gear unit comprises a first gear;
engaging the gripping unit of the gripper with the first holding member of the first unit to restrict the first holding member of the first unit from moving axially with respect to the gripping unit of the gripper but allow the first holding member of the first unit to rotate around the first rotation axis; and
using the first gear of the gear unit of the gripper to rotate the first holding member of the first unit around the first rotation axis, wherein when the first holding member of the first unit rotates around the first rotation axis, the first holding member of the first unit, the gripping unit and the first gear of the gearing unit move axially with respect to the second unit toward or away from the second unit, thereby connecting the first unit with the second unit or disconnecting the first unit from the second unit.

96. The method of claim 95, wherein the automated equipment is a robot.

97. The method of any one of claims 95-96, wherein the first unit comprises a syringe or a connector fixedly coupled with the first holding member.

98. The method of any one of 95-97, wherein the second unit is a connection unit of a self-centering assembly.
